(19) United States
(12) Patent Application Publication
(10) Pub. No.: US 2004/0081708 A1
(43) Pub. Date: Apr. 29, 2004

(54) Methods and compositions for providing glutamine

(76) Inventor: Jeffrey H. Baxter, Galena, OH (US)

Correspondence Address: ROSS PRODUCTS DIVISION OF ABBOTT LABORATORIES, DEPARTMENT 108140-DS/1, 625 CLEVELAND AVENUE, COLUMBUS, OH, 43215-1724, US

(21) Appl. No.: 10/623,194

(22) Filed: Jul. 18, 2003

Related U.S. Application Data
(62) Division of application No. 09/973,105, filed on Oct. 9, 2001.

(51) Int. Cl. A61K 31/70; A61K 31/445; A61K 31/205; A61K 31/495; A61K 31/19; A61K 33/00

(52) U.S. Cl. 424/722; 514/23; 514/563; 514/252.12; 514/316; 514/554; 514/574

(57) ABSTRACT

Methods and compositions for providing glutamine supplementation to a human by orally administering an effective amount of N-acetyl-L-glutamine or a nutritionally acceptable salt thereof. The N-acetyl L-glutamine or a nutritionally acceptable salt thereof can be incorporated into any liquid composition that is suitable for human consumption. Examples of suitable compositions include aqueous solutions such as for use as oral rehydration solutions and liquid nutritional formulas (including enteral formulas, oral formulas, formulas for adults, formulas for children and formulas for infants). The quantity of N-acetyl-L-glutamine or nutritionally acceptable salt thereof can vary widely but typically, these compositions will contain sufficient N-acetyl-L-glutamine or a nutritionally acceptable salt thereof to provide at least 140 mg of total glutamine per kg of body weight per day.

### CROSS REFERENCE

This application is a divisional application of U.S. Patent Application Ser. No. 09/973,105, filed Oct. 9, 2001.

### FIELD OF THE INVENTION

The invention relates to methods for providing glutamine supplementation via the oral administration of an effective amount of N-acetyl-L-glutamine, or a nutritionally acceptable salt thereof.

### BACKGROUND

Glutamine is the most abundant amino acid in the human body. It comprises more than 60% of the free amino acids in skeletal muscle and more than 20% of the total circulating amino acids. Glutamine is involved in many body finctions, including gluconeogenesis, nucleotide synthesis, acid-base balance and other critical metabolic processes. Studies have indicated that glutamine is an important metabolic substrate used by rapidly replicating cells, particularly gastrointestinal tract and mucosal cells. Glutamine can be efficiently absorbed in the human jejunum (part of the small intestine) in vivo.

Glutamine is not considered an essential amino acid because it can be synthesized by virtually all tissues of the body. It is believed to be produced in sufficient quantities to adequately supply body needs (ie., glutamine-consuming tissues) when the body is in a normal physiologic condition. However, numerous studies have shown that during abnormal physiologic conditions (ie., disease and metabolic stress), glutamine production can become insufficient to meet the body's needs. Thus, glutamine may be more accurately considered a conditionally essential amino acid. For example, several studies have classified glutamine as such in cases of gut trauma. Souba, W. W.; Smith, R. J.; and Wilmore, D. J.: Glutamine Metabolism by the Intestinal Tract. JPEN 9(5): 608-617 (1985); Furst, P.; Albers, S and Stehle, P.: Evidence for a nutritional need for glutamine in catabolic patients. Kidney Intl. 36 (Suppl. 27): S-287-S-292 (1989); Klimberg, V. S., et al.: Oral glutamine accelerates healing of the small intestine and improves outcome after whole abdominal radiation. Glutamine has also been suggested as a primary energy source for cultured HeLa cells. Reitzer, L. J.; Wice, B. M.; and Kennell, D.: Evidence that glutamine, not sugar, is the major energy source for cultured HeLa cells. J. Biol. Chem. 254(8): 2669-2676 (1979). And, it has been suggested that glutamine may be preferentially utilized by tumor cells, resulting in progressive glutamine depletion in cancer patients. Souba, W. W.: Glutamine and Cancer. Ann. Surg. 218(6): 715-728 (1993).

Nutritional formulas have previously been supplemented with glutamine. By supplemented it is meant that additional glutamine (either as the free amino acid or in another relatively concentrated form such as hydrolyzed wheat gluten) is added to the formula. As a naturally occurring amino acid, glutamine is present in all proteins to a certain extent, and thus will be present to some extent in any nutritional formula which contains protein. However, glutamine only comprises a certain small amount of most naturally-occurring proteins, and thus, in order to produce a formula with glutamine over a certain level, glutamine must be added in a supplemental form. Some of these glutamine-supplemented formulas are marketed towards patients who are metabolically stressed, who have impaired GI function (such as due to severe multiple trauma, diarrhea, inflammatory bowel disease, GI surgery, severe burns or injury due to chemotherapy or radiation therapy), who have malabsorptive conditions (such as Crohn's disease) and/or acute trauma.

Due to the medical benefits described above, attempts have been made to incorporate glutamine into nutritional products. One problem complicating these efforts is the limited stability of glutamine in aqueous solutions. Free glutamine is known to degrade in aqueous media, forming pyroglutamic acid and glutamic acid. Some studies have shown that pyroglutamic acid is a neurotoxin in rodents. C. F. deMello, et al.: Neurochemical effects of L-pyroglutamic acid. Neurochem. Res. 20(12): 1437-1441 (1995); McGreer, E. G. and Singh, E.: Neurotoxic effects of endogenous materials: quinolinic acid, L-pyroglutamic acid, and thyroid releasing hormone (TRH). Exp. Neurol. 16(3-4): 410-413 (1984); Rieke, G. K., et al.: L-Pyroglutamate: an alternative neurotoxin for a rodent model of Huntington's disease. Exp. Neurol. 104(2): 147-154 (1989). As well as creating pyroglutamic acid, such degradation also decreases the amount of glutamine available for the body when the nutritional formula is fed. Thus, the use of free glutamine as a supplemental glutamine source in nutritional sources has been mostly restricted to powder formulas, which are reconstituted with water immediately or almost immediately (24-48 hours) prior to feeding, and optimally stored under refrigeration after reconstitution. Such powder formulas include AlitraQ® (Ross Products Division of Abbott Laboratories), Nu-Immu® (Enjoy Foods), and Vivonex Plus® (Sandoz). These formulas provide approximately 25.4, 20.1 and 14.5 g of glutamine per 1500 kcal (as analyzed), respectively. Additionally, European Patent Application No. EP 1097646 to Mawatari et al. discloses the use of modified milk powder composition which contains glutamine and/or a peptide containing glutamine. While such products have made a significant contribution to patient care, powdered products are considered less than optimal by most health care facilities in the United States. Due to the shortage of trained medical personnel in many US communities, health care facilities vastly prefer ready-to-feed nutritionals (RTF). Further, these nutritionals must have a shelf-life of at least 12 months to be acceptable in the market place. Thus free glutamine, due to its limited stability, is unacceptable in these RTF products.

Researchers have continued to look for glutamine sources that possess long term stability in solution. For example, U.S. Pat. No. 5,561,111 to Guerrant et al., entitled “Stable Glutamine Derivatives for Oral and Intravenous Rehydration and Nutrition Therapy” discloses the use of alanine-glutamine for this role. Guerrant et al. generically states that acyl protecting groups may be placed on the glutamine, but provides no biological data to substantiate this assertion. Further, this reference fails to provide any guidance on the specific formulation of any oral or intravenous compounds containing such derivatives in such amounts.

This failure is particularly important in light of formulating problems with such solutions as pointed out by Gandini et al., “HPLC Determination of Pyroglutamic Acid as a Degradation Product in Parenteral Amino Acid Formulations” Chromatographia, vol. 36, pp. 75-78 (1993). There, the authors note that in order to overcome the problem of degradation of glutamine into pyroglutamic acid, the use of dipeptides had been proposed but such had the drawback of making the resulting solution qualitatively unbalanced in amino acid content. The authors also note the low bioavailability of the glutamine derivative acetyl-glutamine.

Gurrant et al's lack of biological data is extremely relevant in light of the work of other researchers in this area. Palmerini et al. orally administered radio-labelled N-acetyl-L-glutamine to rats. “Uptake of Doubly-Labelled N-Acetyl-L-Glutamine in Rat Brain and Intestinal Mucosa In Vivo, Farmaco, vol. 36(7), pp. 347-355 (July 1981). Palmerini et al. demonstrated that N-acetyl L-glutamine (NAQ) was absorbed intact across the intestinal mucosa. The lack of intestinal hydrolysis of the acetyl function would lead one skilled in the art to discount NAQ as a potential source of glutamine in nutritional products, since one of glutamine's primary activities is to nourish gut epithelium. This function occurs predominantly during the intestinal absorption of the amino acid.

Disadvantages of using N-acetyl-L-glutamine in nutritional formulas were discussed by Magnusson et al., “Utilization of Intravenously Administered N-Acetyl-L-Glutamine in Humans” Metabolism, vol. 38(8), suppl. 1 (August), pp. 82-88 (1989), who found that 20-40% of the dose of N-acetyl-L-glutamine administered intravenously was excreted in the urine. Other potential problems, especially in rats, were noted by Wallace et al. who concluded that there might be problems with inappetance and inefficient utilization of acetylated peptides, such as N-acetyl-(alanine)₂. “Uptake of acetylated peptides from the small intestine in sheep and their nutritive value in rats” British Journal of Nutrition, v. 80, pp. 101-108 (1998).

### SUMMARY

In accordance with the present invention, it has been discovered that N-acetyl L-glutamine has utility as an oral glutamine supplement in humans. The inventors have discovered that human intestinal tissue can utilize N-acetyl L-glutamine as a source of glutamine. Therefore, N-acetyl-L-glutamine can be incorporated into liquid nutritionals designed for human consumption. These compositions possess long term stability and provide the N-actetyl-L-glutamine in a form that is bioavailable for humans. The N-acetyl L-glutamine may be administered as the acid or as a nutritionally acceptable salt thereof. This finding was unexpected in light of the earlier work done in other mammals besides humans.

The N-acetyl L-glutamine or a nutritionally acceptable salt thereof can be incorporated into any liquid composition that is suitable for human consumption. Examples of suitable compositions include aqueous solutions such as oral rehydration solutions, liquid nutritional formulas (including enteral formulas, oral formulas, formulas for adults, formulas for pediatric patients and formulas for infants), etc. The quantity of N-acetyl L-glutamine or a nutritionally acceptable salt thereof can vary widely but typically, these compositions will contain sufficient N-acetyl-L-glutamine or a nutritionally acceptable salt thereof to provide at least about 10 mg of total glutamine per kg of body weight per day for any human.

### DESCRIPTION OF THE FIGURES

FIG. 1 illustrates in graphic form the aqueous stability of N-acetyl-L-glutamine at various pH values and ambient temperature. All values for pH 5.0 to pH 8.0 samples were the same.

FIG. 2 illustrates in graphic form the degradation products formed in aqueous N-acetyl-L-glutamine solutions over a pH range from 2.0 to 8.0 when the solutions were held at room temperature for 180 days.

FIG. 3 illustrates in graphic form the amount of added glutamine or N-acetyl-L-glutamine remaining in the intestinal lumen as a function of time after introduction of the material to an isolated pig intestinal loop during an Intra-Surgery experiment as described herein. The analyte remaining is expressed as a percentage of the analyte present at time zero.

FIG. 4 illustrates in graphic form the amount of added glucose remaining in the intestinal lumen as a function of time after introduction of the material to an isolated pig intestinal loop during an Intra-Surgery experiment as described herein. Glucose remaining is expressed as a percentage of the amount present at time zero.

FIG. 5 illustrates in graphic form the amount of glutamine in the portal blood (in mcg/mL) in pigs where different materials (glucosaline control, glutamine in glucosaline or N-acetyl-L-glutamine in glucosaline) were introduced to an isolated intestinal loop versus time after administration.

FIG. 6 illustrates in graphic form the amount of glutamine and glutamate in the jejunum mucosa (expressed in mcg/gram wet mucosa) of pig intestine measured after an Intra-Surgery Experiment as described herein

FIG. 7 shows electron transmission micrographs of jejunal mucosa from either healthy or malnourished pigs. Malnourished pigs were fed standard diets (at sub-optimal calorie levels) fortified (at isonitrogenous levels) with glutamine (M-glutamine), N-acetyl-L-glutamine (M-NAQ) or caseinate (M-caseinate). Micrographs were analyzed for signs of inflammation, such as clear cytoplasmic spaces and lymphocyte infiltration.

### DETAILED DESCRIPTION

As used in this application the following terms have the meanings described below:

a) “total glutamine” refers to the total amount of biologically available or potentially available glutamine from any source expressed as glutamine. This can include glutamine supplied as free glutamine, glutamine found as part of a peptide or intact protein, and other biologically available glutamine sources, such as N-acetyl-L-glutamine. Byproducts of glutamine degradation (e.g., pyroglutamic acid and the like) are not included. As an example of this calculation, a hypothetical product is described below.

A nutritional product contains 60 grams/liter of protein system containing intact and lightly hydrolyzed proteins, including the following:

i. Free glutamine at 1.1 grams/liter, as determined by methods well known to one skilled in the art.

ii. A blend of intact and lightly hydrolyzed proteins containing 50.0 grams/liter protein, which has been analyzed by published methodology (e.g., by methods such as Fouques, et al., “Study of the Conversion of Asparagine and Glutamine of proteins into Diaminopropionic and Diaminobutyric Acids Using [Bis(trifluoroacetoxy)iodo] benzene Prior to Amino Acid Determination.” Analyst, Volume 116, (May), pp 529 - 531 (1991)) to contain 3.4 grams glutamine/100 grams protein.

iii. N-Acetyl-L-glutamine at 11.6 grams/liter, which contains 9.0 grams of glutamine as calculated below:

$$11.6 g NAQ \times \frac{1 mole NAQ}{188.2 g NAQ} \times \frac{1 mole Gln}{1 mole NAQ} \times \frac{146.1 g Gln}{1 mole Gln} = 9.0 g Gln$$

“Total Glutamine” is therefore the sum of these three sources, as: 1.1 grams/L (free)+(3.4 g/100 g protein×50 g protein/L)+9.0 grams/L (NAQ)=11.8 grams.

b) “mmoles” refers to millimoles (i.e. 1/1000 of a mole)

c) The term “nutritionally acceptable salt,” means those salts of N-acetyl-L-glutamine which are acceptable for use in a liquid composition that is suitable for administration to humans. Nutritionally acceptable salts of N-acetyl-L-glutamine are salts where the hydrogen of the carboxyl group has been replaced with another positive cation. Such salts can be prepared during the final isolation and purification of the N-acetyl-L-glutamine or separately by reacting the carboxylic group with a suitable base such as the hydroxide, carbonate, or bicarbonate of a metal cation or with ammonia or an organic primary, secondary or tertiary amine. Nutritionally acceptable salt cations may be based on alkali metals or alkaline earth metals such as lithium, sodium, potassium, calcium, magnesium, and aluminum and nontoxic quaternary ammonia and amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine, and piperazine.

d) Any reference in the specification or claims to a quantity of an electrolyte should be construed as referring to the final concentration of the electrolyte in the oral rehydration solution. Tap water often contains residual sodium, chlorine, etc. A value of 40 mEq of sodium, in this application, means that the total sodium present in the oral rehydration solution equals 40 mEq, taking into account both added sodium as well as the sodium present in the water used to manufacture the oral rehydration solution.

e) Any reference to a numerical range in this application should be considered as being modified by the adjective “about”. Further, any numerical range should be considered to provide support for a claim directed to a subset of that range. For example, a disclosure of a range of from 1 to 10 should be considered to provide support in the specification and claims to any subset in that range (i.e. ranges of 2-9, 3-6, 4-5, 2.2-3.6, 2.1-9.9, etc.).

The present invention provides methods and compositions for providing glutamine supplementation to a human by the oral administration of an effective amount of N-acetyl-L-glutamine or a nutritionally acceptable salt thereof. A suitable N-acetyl-L-glutamine for use in the nutritional formulas can be produced using well established, standard chemical synthesis techniques, such as incubating free L-glutamine with acetic anhydride in the presence of a suitable base catylist (e.g., pyridine), following synthesis, suitable purification by recrystallization would produce a suitably pure compound for food-grade status. Indeed, several chemical companies well versed in amino acid chemistries provide a food-grade N-acetyl-L-glutamine (e.g., Kyowa Hakko Kogyo Co, Ltd., Tokyo, Japan or Flamma, s.p.a., Italy). Alternatively, other methods (e.g., microbial fermentation, c.f., JP 51038796, JP 57001994, JP 57016796) could be utilized to produce a suitable food-grade N-acetyl-L-glutamine. Nutritionally acceptable salts of N-acetyl-L-glutamine are salts where the hydrogen of the carboxyl group has been replaced with another positive cation. Such salts can be prepared during the final isolation and purification of the N-acetyl-L-glutamine or separately by reacting the carboxylic group with a suitable base such as the hydroxide, carbonate, or bicarbonate of a metal cation or with ammonia or an organic primary, secondary or tertiary amine. Nutritionally acceptable salt cations may be based on alkali metals or alkaline earth metals such as lithium, sodium, potassium, calcium, magnesium, and aluminum and nontoxic quaternary ammonia and amine cations such as ammonium, tetramethylammonium, tetraethylammonium, methylamine, dimethylamine, trimethylamine, triethylamine, diethylamine, ethylaamine, tributylamine, pyridine, N,N-dimethylaniline, N-methylpiperidine, N-methylmorpholine, dicyclohexylamine, procaine, dibenzylamine, N,N-dibenzylphenethylamine, 1 -ephenamine, and N,N′-dibenzylethylenediamine. Other representative organic amines useful for the formation of base addition salts include ethylenediamine, ethanolamine, diethanolamine, piperidine, and piperazine. If desired pharmacutical grade N-acetyl-glutamine from Sigma may be used.

Methods of providing glutamine supplementation to a human comprises orally administering an effective amount of N-acetyl-glutamine or a nutritionally acceptable salt thereof. Typically, the N-acetyl-L-glutamine will be administered via liquid such as an oral rehydration solution, a sports drink, or a part of an enteral formula.

An effective amount of N-acetyl-glutamine or a nutritionally acceptable salt thereof is preferably an amount sufficient to provide approximately 10-50 g of total glutamine per day or alternatively at least about 140 mg total glutamine per kg of body weight per day, more preferably at least 250 mg total glutamine per kg of body weight per day (mg/kg/day). The N-actetyl-L-glutamine will provide from about 1-100% of the total glutamine that the patient consumes on a daily basis, preferably from about 10-95%, and more preferably from about 75-90% of the total glutamine that the patient consumes on a daily basis.

When N-acetyl-L-glutamine or a nutritionally acceptable salt thereof provides the sole source of glutamine that the patient consumes, an effective amount of N-acetyl-L-glutamine or a nutritionally acceptable salt thereof is preferably at least about 0.7 mmoles/kg/day. More preferably, an effective amount of N-acetyl-L-glutamine or a nutritionally acceptable salt thereof may be at least about 1.0 mmoles/kg/day. Even more preferably, an effective amount of N-acetyl-L-glutamine may be at least about 1.5 mmoles/kg/day.

As noted above, the amount of N-acetyl-L-glutamine or a nutritionally acceptable salt thereof needed to provide total glutamine of 250 mg/kg/day will vary depending upon the amount of glutamine present in any other protein components the patient is consuming. As a general guideline, the patient should consume at least about 7 to about 4.0 mmoles of N-acetyl-L-glutamine or a nutritionally acceptable salt thereof per kg per day to obtain the full benefits of this invention. Lesser amounts may be beneficial, depending on the total glutamine content of the other components of the protein system. In general, sufficient N-acetyl-L-glutamine should be provided to the patient deliver at least about 140 mg of total glutamine per kg of body weight per day, more preferably at least about 250 mg total glutamine per kg of body weight per day.

The method may be utilized to provide glutamine supplementation to adults, children and infants. The term child refers to a human aged one year up to about 16 years( ie adulthood).. The term infant is meant to include all humans less than one year in age, and includes premature infants and micro-preemie infants. The term premature infants is meant to describe infants born before 37 weeks of gestation and/or less than 2500 grams at birth, and the term micro-preemie is meant to describe infants born between 23 and 28 weeks of gestation. As used herein, the term non-adult includes children and infants.

The concentration of glutamine equivalents that is fed to adults, children and infants may vary. One reason for this is the wide variation of caloric density requirements in various stressed situations. One example of this situation arises when only a very small volume of enteral nutrition can be tolerated, such as in severe trauma or in the premature infant. In such cases, the majority of nutrition may initially be provided via parenteral feeding. In these cases, very small amounts of enteral nutrition might be acceptable, and it would be of benefit to supply as much glutamine equivalents as possible. Therefore, a very high concentration of N-acetyl-glutamine or a nutritionally acceptable salt thereof might be used. In another application, a standard infant formulation might be supplemented with N-acetyl-glutamine or a nutritionally acceptable salt thereof to support gut function, in which case a substantially lower concentration would be used The N-acetyl-L-glutamine may be utilizied for any condition in which glutamine may be beneficial. Such conditions include at least: enhanced recovery from gastrointestinal surgery, gastrointestinal resection, small bowel transplant, and other post surgical traumas starvation, critical illnesses and injuries such as multiple trauma, short bowel syndrome, burns, bone marrow transplant, AIDS, oral mucositis, Crohn's disease, necrotizing enterocolitis, prematurity of the gut, and prevention or reduction of severity of infections of opportunity such as sepsis. Glutamine supplementation may also be helpful in preventing gut deterioration associated with particular treatments (such as chemotherapy or radiation therapy) or in situations where oral feeding is severely restricted (such as extreme prematurity). Also included are combinations of any of the above.

The N-acetyl-L-glutamine of this invention can be administered using any liquid solution that is suitable for human consumption. For example, the N-acetyl-L-glutamine may simply be dissolved in water. If desired, it can be incorporated into flavored drinks to enhance its palatability. For example, it can be incorporated into Kool-Aid, or sodas such as Pepsi or Cola. In a further embodiment, the N-acetyl-L-glutamine can be incorporated into sports drinks such as Gator-Aid.

Typically however, the N-acetyl-L-glutamine will be administered via an oral rehydration solution (ORS) or a liquid nutritional formula. The quantity of N-acetyl-L-glutamine that may be incorporated into an aquous solution, such as ORS, can vary widely. Typically, the ORS will contain at least about 5.0 mmoles of N-acetyl-L-glutamine or a nutritionally acceptable salt thereof per liter of solution, and further contain at a minimum, water, glucose, and sodium. More preferably, the ORS will contain about 20 to about 300 mmoles per liter of N-acetyl-L-glutamine or a nutritionally acceptable salt thereof, and more typically from about 25 to about 200 mmoles. If a liquid such as Kool-Aid or Gator-Aid is utilized, then the quantity of N-acetyl-L-glutamine will be comparable to that described for the ORS.

Oral rehydration solutions are well known to those skilled in the art. The ORS's utilized in this invention will typically contain all the electrolytes and levels thereof required by the Food and Drug Administration for oral rehydration formulations sold in the United States. In addition to sodium (Na⁺), potassium (K⁺), chloride (Cl⁻) and citrate ions, the oral rehydration solutions contain a source of carbohydrate, such as glucose, fructose, or dextrose. Typically, the ORS comprise water, carbohydrate, sodium ions, potassium ions, chloride ions, and citrate ions.

The quantity of sodium ions used in the ORS can vary widely, as is known to those skilled in the art. Typically, the ORS will contain from about 30 mEq/L to about 95 mEq/L of sodium. In a further embodiment, sodium content can vary from about 30 mEq/L to about 70 mEq/L, most preferably from about 40 mEq/L to about 60 mEq/L. Suitable sodium sources include but are not limited to sodium chloride, sodium citrate, sodium bicarbonate, sodium carbonate, sodium hydroxide, and mixtures thereof. As used herein, one milliequivalent (mEq) refers to the number of ions in solution as determined by their concentration in a given volume. This measure is expressed as the number of milliequivalents per liter (mEq/L). Milliequivalents may be converted to milligrams by multiplying mEq by the atomic weight of the mineral and then dividing that number by the valence of the mineral.

The ORS will also contain a source of potassium ions. The quantity of potassium can vary widely. However, as a general guideline, the ORS will typically contain from about 10 mEq/L to about 30 mEq/L of potassium. In a further embodiment, they may contain from about 15 mEq/L to about 25 mEq/L of potassium. Suitable potassium sources include, but are not limited to, potassium citrate, potassium chloride, potassium bicarbonate, potassium carbonate, potassium hydroxide, and mixtures thereof.

The ORS will also contain a source of carbohydrate. The quantity of carbohydrate utilized is important as described above. The quantity must be maintained at less than about 3% w/w, and more preferably less than about 2.5% w/w. Quantities ranging from about 3% w/w to about 2.0% w/w are suitable. Excessive carbohydrate will exacerbate the fluid and electrolyte losses associated with diarrhea.

Any carbohydrate used in prior art oral rehydration solutions may be used. Suitable carbohydrates include, but are not limited to, simple and complex carbohydrates, glucose, dextrose, fructoooligosaccharides, fructose and glucose polymers, corn syrup, high fructose corn syrup, sucrose, maltodextrin, and mixtures thereof.

The ORS will also typically include a source of base to replace diarrheal losses. Typically citrate will be incorporated into the oral rehydration solutions to accomplish this result. Citrate is metabolized to an equivalent amount of bicarbonate, the base in the blood that helps maintain acid-base balance. While citrate is the preferred source of base, any base routinely incorporated into rehydration solutions may be used.

The quantity of citrate can vary as is known in the art. Typically, the citrate content ranges from about 10 mEq/L to about 40 mEq/L, more preferably from about 20 mEq/L to about 40 mEq/L, and most preferably from about 25 mEq/L to about 35 mEq/L. Suitable citrate sources include, but are not limited to, potassium citrate, sodium citrate, citric acid and mixtures thereof.

The ORS will also typically contain a source of chloride. The quantity of chloride can vary as is known in the art. Typically the ORS will contain chloride in the amount of from about 30 mEq/L to about 80 mEq/L, more preferably from about 30 mEq/L to about 75 mEq/L, and most preferably from about 30 mEq/L to about 70 mEq/L. Suitable chloride sources include but are not limited to, sodium chloride, potassium chloride and mixtures thereof.

Optionally, indigestible oligosaccharides may be incorporated into the ORS. Indigestible oligosaccharides have a beneficial impact on the microbial flora of the GI tract. They help to suppress the growth of pathogenic organisms such as Clostridium difficile. These oligosaccharides selectively promote the growth of a nonpathogenic microbial flora. Such oral rehydration solutions have been described in U.S. Pat. No. 5,733,759, filed Apr. 5, 1995, the contents of which are hereby incorporated by reference. Typically, the oligosaccharide will be a fructoologosaccharide, an inulin such as raftilose, or a xylooligosaccharide. The quantity can vary widely, but may range from 1 to 100 grams per liter, and more typically from 3 to 30 grams per liter of aqueous solution.

The ORS will also typically include a flavor to enhance its palatability, especially in a pediatric population. The flavor should mask the salty notes of the aqueous solutions. Useful flavorings include, but are not limited to, peach, butter pecan, blueberry, banana, cherry, orange, grape, fruit punch, bubble gum, apple, raspberry and strawberry. Artificial sweeteners may be added to complement the flavor and mask the salty taste. Useful artificial sweeteners include saccharin, nutrasweet, sucralose, acesulfane-K (ace-K), etc.

Preservatives may be added to help extend shelf life. Persons knowledgeable in the art will be able to select the appropriate preservative, in the proper amount, to accomplish this result. Typical preservatives include, but are not limited to, potassium sorbate and sodium benzoate.

In addition to the carbohydrate described above, the ORS may also contain rice flour, or any other component of rice that is beneficial in the treatment of diarrhea. Numerous rice supplemented oral rehydration solutions have been described in the literature. Methods for using such rice supplemented oral rehydration solutions are well known to those skilled in the art. Examples of such rice supplemented oral rehydration solutions include those described in U.S. Pat. No. 5,489,440 issued Feb. 6, 1996; the contents of which are hereby incorporated by reference.

The ORS can be manufactured using techniques well known to those skilled in the art. As a general guideline, all the ingredients may be dry blended together; dispersed in water with agitation; and optionally heated to the appropriate temperature to dissolve all the constituents. The ORS is then packaged and sterilized to food grade standards as is known in the art.

ORS may be administered in different forms, depending upon patient preference, as is known in the art. For example, some children will consume oral rehydration solutions more readily if frozen, such as in the form of a Popsicle. Oral rehydration solution Popsicles are described in detail in U.S. Pat. No. 5,869,459, the contents of which are hereby incorporated by reference. Oral rehydration solutions have also been formed into gels in order to enhance patient compliance, especially in a pediatric population. Gelled rehydration compositions are described in U.S. patent application Ser. No. 09/368,388 filed Aug. 4, 1999, the contents of which are hereby incorporated by reference. These gels have also been described in PCT Application No. 99/15862. As a general overview, the aqueous solutions may be formed into a flowable gel. Alternatively, it may also be formed into a self-supporting gel structure. Such a result may be accomplished by incorporating suitable gelling agents into the aqueous solution.

Suitable gelling agents for use in the aqueous solution include but are not limited to agar, alginic acid and salts, gum arabic, gum acacia, gum talha, cellulose derivatives, curdlan, fermentation gums, furcellaran, gelatin, gellan gum, gum ghatti, guar gum, iota carrageenan, irish moss, kappa carrageenan, konjac flour, gum karaya, lambda carrageenan, larch gum/arabinogalactan, locust bean gum, pectin, tamarind seed gum, tara gum, gum tragacanth, native and modified starch, xanthan gum and mixtures thereof. Usage rates of said gelling agents range from about 0.05 to about 50 wt./wt. %.

As noted above, the N-acetyl-L-glutamine, or its nutritionally acceptable salts may be administered via liquid nutritional products. The quantity of N-acetyl-glutamine that is incorporated into the liquid nutritional can vary widely, but will fit into the dosage guidelines described above. The amount of N-acetyl-L-glutamine or a nutritionally acceptable salt thereof utilized in a liquid nutritional formula will be dependent upon various factors including whether the formula provides a majority or sole source of nutrition, whether the formula contains other sources of glutamine, the amount of formula consumed on a daily basis, and the type of patient for whom the formula is intended (which will also influence the amount of formula consumed daily). The formula will preferably contain N-acetyl-L-glutamine or a nutritionally acceptable salt thereof in an amount sufficient, when combined with the glutamine contained in the other protein components, to provide at least 140 mg of total glutamine per kg of body weight per day. The amount of N-acetyl-L-glutarnine or a nutritionally acceptable salt thereof may also be expressed as providing a percentage of the protein calories. According to such an expression, nutritional formulas would contain N-acetyl-L-glutamine or a nutritionally acceptable salt thereof as about 1 to about 100% of the protein calories. The percentages are calculated based on the protein portion of N-acetyl-L-glutamine or a nutritionally acceptable salt thereof (ie., the glutamine portion), and do not take into account any caloric contribution from the non-protein portion of N-acetyl-glutamine or a nutritionally acceptable salt thereof (ie., the acetate or salt portion). Preferably, when a nutritional formula is for adults, it would contain N-acetyl-L-glutamine or a nutritionally acceptable salt thereof sufficient to supply about 10 to about 95% of the protein calories. If the nutritional formula is being designed for non-adults, then the N-acetyl-L-glutamine would be present in sufficient quantities to supply from about 1 to about 12% of the protein calories.

Liquid nutritional formulas include enteral formulas, oral formulas, formulas for adults, formulas for pediatric patients and formulas for infants. Enteral formulas and nutritional formulas, for example, represent an important component of patient care in both acute care hospitals and long term care facilities (ie., nursing homes). These formulas can serve as the sole source of nutrition for a human being over an extended period of time, though supplemental use to enhance sub-optimal nutrition status is common. Accordingly, the formulas must contain significant amounts of protein, fat, minerals, electrolytes, etc., if they are to meet their primary goal of preventing malnutrition. These formulas are typically administered to the patient as a liquid, since a significant proportion of the patients targeted are incapable of consuming solid foods. While some patients are capable of drinking a formula, there are significant numbers that receive enteral formulas via a nasogastric tube (NG tube or tube feeding).

Liquid nutritional formulas contain a protein component, providing from 14 to 35% of the total caloric content of the formula, a carbohydrate component providing from 36 to 76% of the total caloric content, and a lipid component providing from 6 to 51% of the total caloric content. Liquid nutritional formulas may be adult formulas, pediatric formulas or infant formulas (Oust as the aqueous solutions may be administered to either adults, pediatric patients or infants). For high glutamine applications, liquid nutritional formulas preferably provide at least a majority source of nutrition. The liquid nutritional formulas described herein, however, may be used as other than an at least majority source of nutrition, particularly in the case where mostly parenteral nutrition is the standard of practice (e.g., in extremely premature infants, who are slowly weaned to oral feedings over the first several weeks ex utero). The term at least a majority source of nutrition means that the formula is intended to be fed in an amount sufficient to provide at least half of the total caloric and nutritional requirements for a patient receiving the formula. Encompassed within this definition are formulas and the feeding of formulas as a sole source of nutrition, thereby providing all of the total caloric and nutritional requirements for a patient receiving the formula. The amount of calories and nutrients required will vary from patient to patient, dependent upon such variables as age, weight, and physiologic condition. The amount of nutritional formula needed to supply the appropriate amount of calories and nutrients may be determined by one of ordinary skill in the art, as may the appropriate amount of calorie and nutrients to incorporate into such formulas. As examples, when the formula is an adult formula, the protein component may comprise from about 14 to about 35% of the total caloric content of said liquid nutritional formula; the carbohydrate component may comprise from about 36 to about 76% of the total caloric content of said liquid nutritional formula; and the lipid component may comprise from about 6 to about 41% of the total caloric content of said liquid nutritional formula. The nutritional formula may be a formula for oral feeding or a formula for enteral feeding. As another example, when the formula is a non-adult formula, the protein component may comprise from about 8 to about 25% of the total caloric content of said liquid nutritional formula; the carbohydrate component may comprise from about 39 to about 44% of the total caloric content of said liquid nutritional formula; and the lipid component may comprise from about 45 to about 51% of the total caloric content of said liquid nutritional formula. These ranges are provided as examples only, and are not intended to be limiting.

As a practical matter, such products would contain an amount of N-acetyl-L-glutamine or a nutritionally acceptable salt thereof sufficient to provide about half or more of the total glutamine content. Alternatively, an effective amount of N-acetyl-L-glutamine or a nutritionally acceptable salt thereof may be expressed in mmoles per 1000 kcal. According to such an expression, if a target amount of glutamine is approximately 300 mg of glutamine per day/kg/day, a nutritional formula would preferably contain for an adult, at least about 35 mmoles of N-acetyl-L-glutamine or a nutritionally acceptable salt thereof per 1000 kcal of nutritional formula, and for a child, infant or premature infant (a non-adult) at least about 5.0 mmoles of N-acetyl-L-glutamine or a nutritionally acceptable salt thereof per 1000 kcal of nutritional formula. More preferably, such nutritional formula for an adult would contain about 35 to about 160 mmoles of N-acetyl L-glutamine or a nutritionally acceptable salt thereof per 1000 kcal of nutritional formula, for a child about 5.0 to about 32 mmoles of N-acetyl-L-glutamine or a nutritionally acceptable salt thereof per 1000 kcal of nutritional formula, and for an infant or premature infant about 5.0 to about 26 mmoles of N-acetyl-L-glutamine or a nutritionally acceptable salt thereof per 1000 kcal of nutritional formula.

In addition to the N-acetyl-glutamine, the nutritional formulas will contain suitable carbohydrates, lipids and proteins as is known to those skilled in the art of making nutritional formulas. Suitable carbohydrates include, but are not limited to, hydrolyzed, intact, naturally and/or chemically modified starches sourced from corn, tapioca, rice or potato in waxy or non waxy forms; and sugars such as glucose, fructose, lactose, sucrose, maltose, high fructose corn syrup, corn syrup solids, fructooligosacchardies, and mixtures thereof. Maltodextrins are polysaccharides obtained from the acid or enzyme hydrolysis of starches (such as those from corn or rice). Their classification is based on the degree of hydrolysis and is reported as dextrose equivalent (DE). The DE of any maltodextrins utilized in the nutritional formulas is preferably less than about 18-20.

Suitable lipids include, but are not limited to, coconut oil, soy oil, corn oil, olive oil, safflower oil, high oleic safflower oil, MCT oil (medium chain triglycerides), sunflower oil, high oleic sunflower oil, palm oil, palm olein, canola oil, cottonseed oil, fish oil, palm kernel oil, menhaden oil, soybean oil, lecithin, lipid sources of arachidonic acid and docosahexaneoic acid, and mixtures thereof. Lipid sources of arachidonic acid and docosahexaneoic acid include, but are not limited to, marine oil, egg yolk oil, and fungal or algal oil. Numerous commercial sources for these fats are readily available and known to one practicing the art. For example, soy and canola oils are available from Archer Daniels Midland of Decatur, Ill. Corn, coconut, palm and palm kernel oils are available from Premier Edible Oils Corporation of Portland, Oreg. Fractionated coconut oil is available from Henkel Corporation of LaGrange, Ill. High oleic safflower and high oleic sunflower oils are available from SVO Specialty Products of Eastlake, Ohio. Marine oil is available from Mochida International of Tokyo, Japan. Olive oil is available from Anglia Oils of North Humberside, United Kingdom. Sunflower and cottonseed oils are available from Cargil of Minneapolis, Minn. Safflower oil is available from California Oils Corporation of Richmond, Calif.

In addition to these food grade oils, structured lipids may be incorporated into the nutritional if desired. Structured lipids are known in the art. A concise description of structured lipids can be found in INFORM, Vol.. 8, no. 10, page 1004, entitled Structured lipids allow fat tailoring (October 1997). Also see U.S. Pat. No. 4,871,768 which is hereby incorporated by reference. Structured lipids are predominantly triacylglycerols containing mixtures of medium and long chain fatty acids on the same glycerol nucleus. Structured lipids and their use in enteral formula are also described in U.S. Pat. Nos. 6,194,37 and 6,160,007, the contents of which are hereby incorporated by reference.

Suitable protein sources include, but not limited to, milk, whey and whey fractions, soy, rice, meat (e.g., beef), animal and vegetable (e.g., pea, potato), egg (egg albumin), gelatin and fish. Suitable intact protein sources include, but are not limited to, soy based, milk based, casein protein, whey protein, rice protein, beef collagen, pea protein, potato protein, and mixtures thereof. Suitable protein hydrolysates include, but are not limited to, soy protein hydrolysate, casein protein hydrolysate, whey protein hydrolysate, rice protein hydrolysate, potato protein hydrolysate, fish protein hydrolysate, egg albumen hydrolysate, gelatin protein hydrolysate, a combination of animal and vegetable protein hydrolysates, and mixtures thereof. Hydrolyzed proteins (protein hydrolysates) are proteins that have been hydrolyzed or broken down into shorter peptide fragments and amino acids. Such hydrolyzed peptide fragments and free amino acids are more easily digested. In the broadest sense, a protein has been hydrolyzed when one or more amide bonds have been broken. Breaking of amide bonds may occur unintentionally or incidentally during manufacture, for example due to heating or shear. For purposes of this disclosure, hydrolyzed protein means a protein which has been processed or treated in a manner intended to break amide bonds. Intentional hydrolysis may be effected, for example, by treating an intact protein with enzymes or acids. The hydrolyzed proteins that are preferably utilized in the liquid nutritional formulas described herein are hydrolyzed to such an extent that the ratio of amino nitrogen (AN) to total nitrogen ranges from about 0.1 AN to about 1.0 TN to about 0.4 AN to about 1.0 TN, preferably about 0.25 AN to 1.0 TN to about 0.4 AN to about 1.0 TN. (AN:TN ratios are given for the hydrolysate protein alone and do not represent the AN:TN ratios in the final nutritional formulas.)

Protein may also be provided in the form of free amino acids. The nutritional formulas may be supplemented with various amino acids in order to provide a more nutritionally complete and balanced formula. Examples of suitable free amino acids include, but are not limited to, all free L-amino acids usually considered a part of the protein system, but especially those considered essential or conditionally essential from a nutritional standpoint, namely: tryptophan, tyrosine, cyst(e)ine, methionine, arginine, leucine, valine, lysine, phenylalanine, isoleucine, threonine, and histidine. Other (non-protein) amino acids typically added to nutritional products include carnitine and taurine. In some cases, the D-forms of the amino acids are considered as nutritionally equivalent to the L-forms, and isomer mixtures are used to lower cost (for example, D,L-methionine).

The nutritional formulas preferably also contain vitamins and minerals in an amount designed to supply the daily nutritional requirements of the patient receiving the formula. Those skilled in the art recognize that nutritional formulas often need to be over fortified with certain vitamins and minerals to ensure that they meet the daily nutritional requirements over the shelf life of the product. These same individuals also recognize that certain microingredients may have potential benefits for people depending upon any underlying illness or disease that the patient is afflicted with. For example, diabetics benefit from such nutrients as chromium, carnitine, taurine and vitamin E. Formulas preferably include, but are not limited to, the following vitamins and minerals: calcium, phosphorus, sodium, chloride, magnesium, manganese, iron, copper, zinc, selenium, iodine, chromium, molybdenum, conditionally essential nutrients m-inositol, camitine and taurine, and Vitamins A, C, D, E, K and the B complex, and mixtures thereof.

If the liquid nutritional is intended for an infant, then specific nutritional guidelines for may be found in the Infant Formula Act, 21 U.S.C. section 350(a). The nutritional guidelines found in these statutes continue to be refined as further research concerning nutritional requirements is completed. The nutritional formulas claimed are intended to encompass formulas containing vitamins and minerals that may not currently be listed.

The liquid nutritional formulas also may contain fiber and stabilizers. Suitable sources of fiber/and or stabilizers include, but are not limited to, xanthan gum, guar gum, gum arabic, gum ghatti, gum karaya, gum tracacanth, agar, furcellaran, gellan gum, locust bean gum, pectin, low and high methoxy pectin, oat and barley glucans, carageenans, psyllium, gelatin, microcyrstalline cellulose, CMC (sodium carboxymethylcellulose), methylcellulose hydroxypropyl methyl cellulose, hydroxypropyl cellulose, DATEM (diacetyl tartaric acid esters of mono- and diglycerides), dextran, carrageenans, FOS (fructooligosaccharides), and mixtures thereof. Numerous commercial sources of soluble dietary fibers are available. For example, gum arabic, hydrolyzed carboxymethylcellulose, guar gum, pectin and the low and high methoxy pectins are available from TIC Gums, Inc. of Belcamp, Md. The oat and barley glucans are available from Mountain Lake Specialty Ingredients, Inc. of Omaha, Nebr. Psyllium is available from the Meer Corporation of North Bergen, N.J. while the carrageenan is available from FMC Corporation of Philadelphia, Pa.

The fiber incorporated may also be an insoluble dietary fiber representative examples of which include oat hull fiber, pea hull fiber, soy hull fiber, soy cotyledon fiber, sugar beet fiber, cellulose and corn bran. Numerous sources for the insoluble dietary fibers are also available. For example, the corn bran is available from Quaker Oats of Chicago, Ill.; oat hull fiber from Canadian Harvest of Cambridge, Minn.; pea hull fiber from Woodstone Foods of Winnipeg, Canada; soy hull fiber and oat hull fiber from The Fibrad Group of LaVale, Md.; soy cotyledon fiber from Protein Technologies International of St. Louis, Mo.; sugar beet fiber from Delta Fiber Foods of Minneapolis, Minn. and cellulose from the James River Corp. of Saddle Brook, N.J.

A more detailed discussion of examples of fibers and their incorporation into formula may be found in U.S. Pat. No. 5,085,883 issued to Garleb et al which is hereby incorporated by reference.

The quantity of fiber utilized in the formulas can vary. The particular type of fiber that is utilized is not critical. Any fiber suitable for human consumption and that is stable in the matrix of a nutritional formula may be utilized.

In addition to fiber, the nutritionals may also contain oligosaccharies such as fructooligosaccharies (FOS) or glucooligosacchairdes (GOS). Oligosaccharides are rapidly and extensively fermented to short chain fatty acids by anaerobic microorganisms that inhabit the large bowel. These oligosaccharides are preferential energy sources for most Bifidobacterium species, but are not utilized by potentially pathogenic organisms such as Clostridium perfingens, C. difficile, or E. coli.

The liquid nutritional formulas may also contain a flavor to enhance its palatability. Useful flavorings include, but are not limited to, chocolate, vanilla, coffee, peach, butter pecan, blueberry, banana, cherry, orange, grape, fruit punch, bubble gum, apple, raspberry and strawberry. Artificial sweeteners may be added to complement the flavor and mask salty taste. Useful artificial sweeteners include saccharin, nutrasweet, sucralose, acesulfane-K (ace-K), etc..

Liquid nutritional formulas can be manufactured using techniques well known to those skilled in the art. Various processing techniques exist. Typically these techniques include formation of a slurry from one or more solutions which may contain water and one or more of the following: carbohydrates, proteins, lipids, stabilizers, vitamins and minerals. The slurry is emulsified, homogenized and cooled. Various other solutions may be added to the slurry before processing, after processing or at both times. The processed formula is then sterilized and may be diluted to be utilized on a ready-to-feed basis or stored in a concentrated liquid form. When the resulting formula is meant to be a ready-to-feed liquid or concentrated liquid, an appropriate amount of water would be added before sterilization.

### EXAMPLES

### Method for Preparing Liquid Nutritional Formulas

Liquid nutritional formulas falling within the scope of the claims can be prepared by the following procedures. These examples are being presented as illustrations and should not be interpreted as limiting. Other carbohydrates, lipids, proteins, stabilizers, vitamins and minerals may be used without departing from the scope of the invention.

### Example 1

### Method for Preparing Liquid Nutritional Formulas Containing N-acetyl-L-glutamine

A ready-to-feed liquid product was made containing N-acetyl-L-glutamine using the materials listed in Table 1. The procedure used to produce the product is outlined below.

TABLE 1
Bill of Materials for Vanilla Flavored Product

| | Amount |
|---|---|
| Ingredient Name | (per 1000 kg) |
| Water | to final mass |

| | | |
|---|---|---|
| Maltodextrin | 77.88 | kg |
| Sucrose | 52.80 | kg |
| Soy Protein Hydrolysate | 30.11 | kg |
| Fish oil/Medium Chain Structured lipid | 16.14 | kg |
| sodium caseinate | 14.74 | kg |
| Fructooligosaccharide | 5.792 | kg |
| Canola oil | 4.842 | kg |
| Soybean oil | 4.842 | kg |
| 45% Potassium Hydroxide | 3.653 | kg |
| Tri-calcium Phosphate | 2.866 | kg |
| N-Acetyl-L-glutamine | 10.03 | kg |
| L-Arginine | 2.425 | kg |
| Sodium citrate | 2.293 | kg |
| Artificial Carmel | 1.500 | kg |
| N&A Vanilla Flavor | 1.000 | kg |
| Emulsifier | 1.076 | kg |
| Magnesium phosphate | 0.948 | kg |
| Magnesium chloride | 0.860 | kg |
| Potassium citrate | 0.838 | kg |
| Ascorbic acid | 0.697 | kg |
| Choline chloride | 0.474 | kg |
| Gellan gum | 0.250 | kg |
| Vitamin D, E, K Premix¹ | 0.203 | kg |
| Taurine | 0.139 | kg |
| Carnitine | 0.130 | kg |
| Vitamin E (R,R,R) (81%) | 0.123 | kg |
| Trace Mineral Premix² | 0.101 | kg |
| Water Soluble Vitamin Premix³ | 0.0882 | kg |
| 30% beta Carotene | 15.5 | grams |
| Vitamin A (55%) | 5.07 | grams |
| Potassium Iodide | 0.194 | grams |
| Sodium Selenite | 0.132 | grams |
| Vitamin K | 0.0617 | grams |

¹The vitamin D, E, K premix includes vitamin D3 (0.0980 grams), d-alpha-tocopheryl acetate (55.93 grams), and vitamin K1 (0.0338 grams) in a coconut oil (146.77 grams) carrier.
²The trace mineral premix delivers (per 1000 kg Finished Product) zinc sulfate (46.3 grams), ferrous sulfate (39.2 grams), manganese sulfate (11.4 grams), copper sulfate (3.89 grams).
³The water soluble vitamin premix includes niacinamide (33.07 grams), d- calcium pantothenate (21.43 grams), folic acid (0.742 grams), thiamine chloride HCL (5.47 grams), riboflavin (4.27 grams), pyroxidine HCL (5.26 grams), cyanocobalamin (0.0147 grams) and biotin (0.644 grams) in a dextrose (17.29 grams) carrier.

PROCEDURE: The liquid nutritional product described above is manufactured by preparing three slurries which are blended together, combined with the marine oil/MCT structured lipid, heat treated, standardized, packaged and sterilized. A process for manufacturing is described in detail below.

A carbohydrate/mineral slurry is prepared by first heating an appropriate amount of water to a temperature between about 65° C. and about 71° C. with agitation. The required amount of minerals are then added in the order listed, under high agitation: sodium citrate, trace mineral premix, potassium citrate, magnesium chloride, magnesium phosphate, tricalcium phosphate and potassium iodide. Next, the required amount of maltodextrin (Maltring M-100 distributed by Grain Processing Corporation of Muscatine, Iowa) is added to the slurry under high agitation, and is allowed to dissolve while the temperature is maintained at about 71° C. The required amount of sucrose and Fructooligosaccharide (Nutriflora-P® Fructo-oligosaccharide Powder distributed by Golden Technologies Company of Golden, Colo.) are then added under high agitation. The required amount of gellan gum (Kelcogel® distributed by Kelco, Division of Merck and Company Incorporated of San Diego, Calif.) is then dry blended with sucrose in a 1:5 (gellan gum/sucrose ratio), and added to the slurry under high agitation. Next, sodium selenite that has been dissolved in warm water is added to the slurry under agitation. The completed carbohydrate/mineral slurry is held with high agitation at a temperature between about 65° C. and about 71° C. for not longer than twelve hours until it is blended with the other slurries.

An oil blend is prepared by combining and heating the required amounts of soybean oil and canola oil to a temperature between about 55° C. and about 65° C. with agitation. The required amount of emulsifier, diacetyl tartaric acid esters of monodiglycerides, (Panodan® distributed by Grindsted Products Incorporated of New Century, Kans.) is then added under agitation and allowed to dissolve. The Vitamin D, E, K premix, 55% Vitamin A Palmitate, D-alpha-a-tocopherol acetate (R,R,R form), phylloquinone and 30% beta-carotene are then added with agitation. The completed oil blend is held under moderate agitation at a temperature between about 55° C. and about 65° C. for a period of no longer than twelve hours until it is blended with the other slurries.

A protein in water slurry is prepared by first heating an appropriate amount of water to a temperature between about 60° C. and about 71° C. with agitation. Soy protein hydrolysate (distributed by MD Foods of Viby J., Denmark) is added with agitation. The required amount of N-acetyl-L-glutamine (obtained from Ajinomoto) is added with agitation. Potassium hydroxide solution (45%) is added to raise pH to about 5.6. L-arginine is slowly added, with agitation, and the solution stirred until clarified (pH >6.2). The required amount of partially hydrolyzed sodium caseinate (Alanate® 167 distributed by New Zealand Milk Products Incorporated of Santa Rosa, Calif.) is then blended into the slurry. This completed protein-in-water slurry is held under moderate agitation at a temperature between about 60° C. and about 71° C. for a period of no longer than two hours until it is blended with the other slurries.

The protein-in-water slurry and oil blend are mixed with agitation and the resultant blended slurry is maintained at a temperature between about 55° C. and about 65° C. After waiting for at least one minute, the carbohydrate/mineral slurry is added with agitation and the resultant blended slurry is maintained at a temperature between about 55° C. and about 65° C. The marine oil/MCT structured lipid is then added to the blended slurry with agitation. Desirably, the marine oil/MCT structured lipid is slowly metered into the product as the blend passes through a conduit at a constant rate. After waiting for a period of not less than one minute nor greater than two hours, the blend slurry is subjected to deaeration, ultra-high-temperature treatment, and homogenization, using techniques known to one skilled in the art. The blend is then cooled to a temperature between about 1° C. and about 7° C., stored at a temperature between about 1° C. and about 7° C. with agitation. Preferably, after the above steps have been completed, appropriate analytical testing for quality control is conducted. Based on the analytical results of the quality control tests, an appropriate amount of water is added to the batch with agitation for final dilution (standardization).

The vitamin solution is prepared by heating a small amount of water to a temperature between about 43° C. and about 66° C. with agitation, and thereafter adding the following ingredients with agitation: ascorbic acid, 45% potassium hydroxide, taurine, water soluble vitamin premix, choline chloride, and L-camitine. The vitamin slurry is then added to the blended slurry under agitation.

A flavor solution is prepared by adding the natural and artificial vanilla flavor and artificial caramel flavor to an appropriate amount of water with agitation. The flavor slurry is then added to the blended slurry under agitation.

The product pH may be adjusted to achieve optimal product stability. The completed product is then placed in suitable containers (in this case, 8 oz. metal cans) and subjected to terminal sterilization (in this case, retort sterilization).

### Example 2

### Aqueous N-acetyl-L-glutamine Stability Studies

Studies were conducted to assess the stability of aqueous N-acetyl-glutamine upon heating, at various pH values, and in a matrix similar to that found in a liquid nutritional type product.

### Aqueous N-acetyl-L-glutamine and Glutamine Heat Stability

In order to test the stability of aqueous N-acetyl-glutamine upon heating, the following procedure was followed. Aqueous solutions of N-acetyl-L-glutamine (obtained from Sigma, catalog no. A-9125) and glutamine (obtained from Aldrich, catalog no. G-320-2) at approximately 1 mg/mL (5.3 mM and 6.8 mM, respectively) were prepared without pH adjustment. The pH of the resulting N-acetyl-L-glutamine solution was 2.9 and the pH of the glutamine solution was 6.0. The solutions were heated at 100° C. using a Reacti-Therm stirring heat block with sealed 4 mL vessels, one for each time point: 15 minutes, 30 minutes, 1 hour and 2 hours. The samples were removed from the heat block and immediately placed into ice until cool. An aliquot of each sample was filtered through 0.45 micrometer filters (Millipore Millex-HV, 25 mm) for assessment by HPLC.

HPLC analysis was conducted using an Inertsilt C8, 5 micrometer, 4.6×250 mm column (obtained from Keystone Scientific, Inc., Bellefonte, Pa.). The mobile phase was water adjusted to pH 2.2 with HCl (isocratic at 1 mL/minute). The injection volume was 10 microliters. Ultraviolet detection was at 214 nm.

Results are provided in Table 2. Glutamine was not stable during the 2 hour incubation at 100° C. The major degradation product after boiling the pH 6.0 glutamine solution for 1 hour was pyroglutamic acid. After boiling the glutamine solution for 2 hours, pyroglutamic acid was still the major degradation product, but glutamic acid was also detected.

N-acetyl-L-glutamine was much more stable than glutamine. The major degradation product was tentatively identified by retention time as N-acetyl-glutamic acid; this identification was confirmed by mass spectrometry (MS) and nuclear magnetic resonance spectrometry (NMR). The second largest peak, as identified by MS and NMR, was 2, 6-dioxopiperidinylacetamide. In the N-acetyl-L-glutamine solutions, pyroglutamic acid was detected only in the 2 hour sample, and only at the very low level of 0.2 area percent.

TABLE 2
Aqueous Solution of Glutamine and N-acetyl-L-glutamine heated at 100° C.

| | Glutamine solution | | | N-acetyl-L-glutamine solution | | | |
|---|---|---|---|---|---|---|---|
| | (height %) | | | (area %) | | | |
| Time | | | | | | | |
| (min.) | GLN¹ | GLU² | PGA³ | NAQ⁴ | 2,6-DPA⁵ | PGA³ | NAE⁶ |
| 0 | 100.0 | — | none detected | 99.7 | — | — | 0.3 |
| 30 | 90.2 | — | 9.8 | 98.1 | 0.6 | — | 1.2 |
| 60 | 80.0 | — | 20.0 | 96.9 | 1.3 | — | 1.8 |
| 120 | 53.6 | 10.6 | 35.8 | 93.4 | 2.7 | 0.2 | 3.7 |

¹glutamine, ²glutamate, ³pyroglutamic acid, ⁴N-acetyl-L-glutamine, ⁵2,6-dioxopiperidinylacetamide ⁶N-acetyl-L-glutamic acid

### Aqueous N-acetyl-L-glutamine and Glutamine Stability at Various pH Values

In order to test the stability of N-acetyl-L-glutamine in aqueous solutions at various pH values, the following procedure was followed. Aqueous solutions of N-acetyl-L-glutamine were prepared in 1 pH unit increments from pH 2.0 to 8.0. The pH of the solutions was adjusted with either hydrochloric acid or sodium hydroxide, as needed, just prior to final dilution (final concentration=1 mg/mL or 5.3 mM N-acetyl-L-glutamine). A single solution of glutamine was not pH adjusted (measured pH=6.0) and was prepared at 1 mg/mL or 6.8 mM glutamine. All solutions were sterile-filtered (Millipore Millex-GS, 25 mm, 0.22 micrometer pore size, sterile) into autosampler vials and capped for storage at ambient temperature (17-25° C.). N-acetyl-L-glutamine samples were assessed by HPLC at various time points, from 1 to 180 days. The glutamine sample was assessed by HPLC at similar time points, from 1 to 45 days.

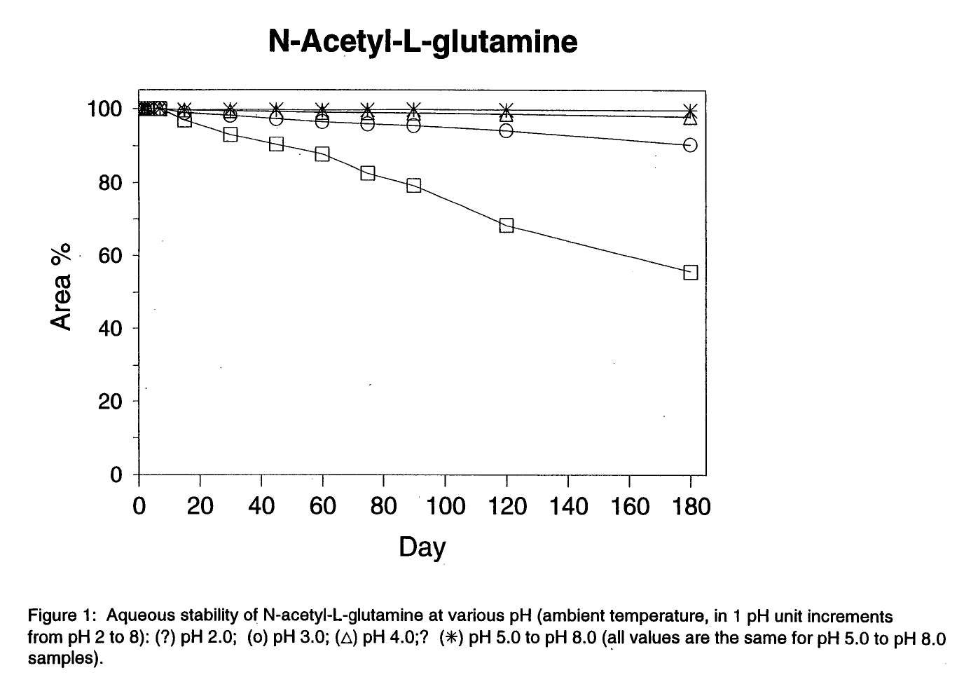

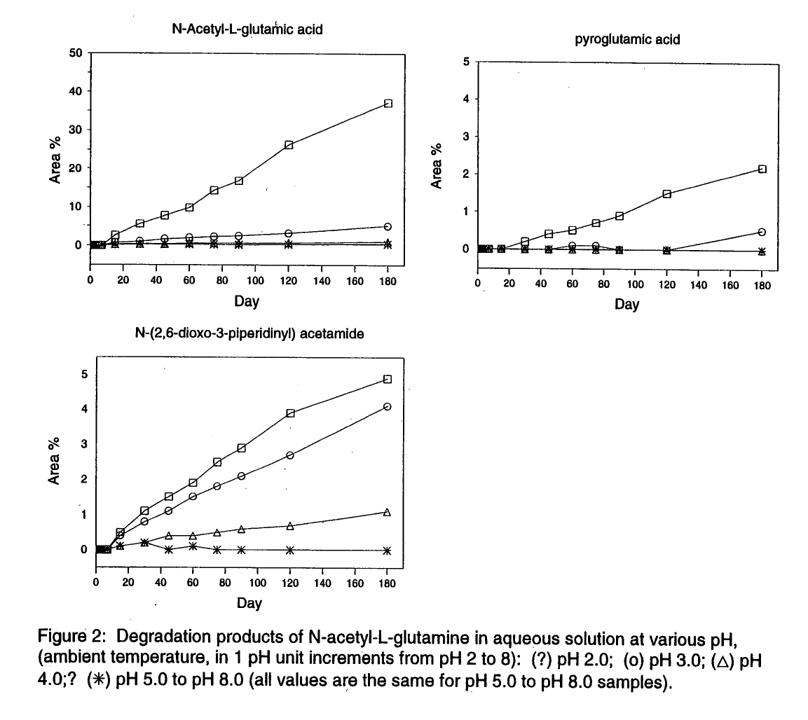

The stability of N-acetyl-L-glutamine was found to be pH dependent. Results are reported in FIGS. 1 and 2. At all pH values, N-acetyl-L-glutamine showed no degradation through 7 days. At pH 5.0 to 8.0, N-acetyl-L-glutamine was stable over 6 months; greater than 99.6% of N-acetyl-L-glutamine remained. The only consistently detected degradation product was N-acetyl-glutamic acid at less than 0.5% through six months. At pH 4.0, by six months, each of N-acetyl-glutamic acid and 2, 6-dioxopiperidinylacetamide was detected with 97.9% N-acetyl-L-glutamine remaining. At pH 3.0, N-acetyl-L-glutamine remained at >95% through 90 days, dropping to 94.2% at 4 months and 90.4% at 6 months. N-acetyl-glutamic acid and 2,6-dioxopiperidinylacetamide were detected at approximately equal levels in the pH 3.0 samples starting at about 0.15% at 15 days, increasing to about 1% at 30 days and about 5% at 6 months. At 6 months, pyroglutamic acid was detected at 0.5%. At pH 2.0, N-acetyl-L-glutamine was 97.0% at 15 days, but decreased to only 55.7% at 6 months. N-acetyl-glutamic acid was the major degradation product in the pH 2.0 sample, at 2.5% in the 15 day sample and 37.2% in the 6 months sample. 2, 6-dioxopiperidinyl acetamide increased from 0.5% at 15 days to 4.9% at 6 months. The pH 2.0 N-acetyl-L-glutamine sample was the only sample that showed increasing values for pyroglutamic acid: 0.2% at 30 days to 2.2% at 6 months.

In the glutamine solution (pH 6.0), pyroglutamic acid was found in the sample after 3 days at room temperature at 0.2%. After 45 days, it was found at 3.3% and glutamine was at 96.7%. Results from HPLC analysis are reported as height percent in Table 3.

TABLE 3
Stability of Glutamine in pH 6.0 Aqueous Solution
At 1 mg/mL and Ambient Temperature.

| Analyte | 2 days | 3 days | 7 days | 15 days | 30 days | 45 days |
|---|---|---|---|---|---|---|
| GLN¹ | 100.0 | 99.8 | 99.5 | 99.0 | 98.1 | 96.7 |
| PGA² | none detected | 0.2 | 0.5 | 1.0 | 1.9 | 3.3 |

¹glutamine, ²pyroglutamic acid

### N-acetyl-L-glutamine and Glutamine Stability in Liquid Nutritional Type Products

In order to test the stability of N-acetyl-L-glutamine in a matrix similar to that found in liquid nutritional type products, the following procedure was followed. Three study products were formulated, one containing N-acetyl-L-glutamine ( N-acetyl-L-glutamine was obtained from Ajinomoto), one containing glutamine (obtained from Ajinomoto) (at theoretical concentrations of 16.5 mg/mL and 12.8 mg/mL, respectively, and replacing part of the protein on a weight basis), and a control (Optimental®, Ross Products Division, Abbott Laboratories). The product containing N-acetyl-L-glutamine was made according to the procedure set forth above in Example 1. The product containing glutamine was made in a similar manner, except glutamine (7.79 kg) was substituted for N-acetyl-L-glutamine. The products were assessed for degradation before and after a retort sterilization process, which is typical for liquid nutritional processing (here, 128° C. for 5 minutes). The products were stored at room temperature (20-22° C.) and assessed for evidence of degradation at 1, 2 and 3 months. Glutamine, N-acetyl-L-glutamine and pyroglutamic acid (if present) were quantified at each process and time point.

In order to analyze by HPLC for glutamine, N-acetyl-L-glutamine and pyroglutamic acid, samples were filtered as follows. A 5.0 mL aliquot was transferred to a 50 mL volumetric flask. Twenty drops of 1 M hydrochloric acid was added and the sample was diluted to volume with deionized water. An aliquot was filtered through a 0.45 micron filter (Millipore, Millex-HV, 25 mm). The samples were analyzed by HPLC as described above (Heat Stability section).

The total amount of pyroglutamic acid present in the protein formula, including both free pyroglutamic acid and N-terminal pyroglutamic acid, can be determined by the following method. Initially, samples were prepared as a water solution to a concentration of approximately 18 g total protein/L. A 20 microliter aliquot of the prepared sample material was placed in a 1.5 mL screw cap vial, and 980 microliters of a freshly prepared enzyme solution (0.05 M Tris, 0.005 M dithiothreitol, 0.001 M disodium ethylenediaminetetraacetic acid (EDTA), pH 8.0, containing 11 units of pyroglutamate aminopeptidase/mL) was added. The vial was tightly capped, and incubated at room temperature (21-24° C.) for 24 hours. The solution was then processed through a C-18 SPE cartridge as detailed below. For free pyroglutamic acid determination, the initial sample solution was diluted to a total protein content of 2-3 g/L in deionized water, and processed through a C-18 SPE cartridge.

C-18 SPE (Solid Phase Extraction) cartridges (100 mg/1 mL size) were obtained from Burdick & Jackson, Muskegon, Mich. SPE cartridges were prepared for use with 2×5 volumes of methanol, and then rinsed with 2×5 volumes of deionized water. The 1 mL sample is then slowly applied, and flow-through material collected in a 1 dram screw cap vial. Elution was completed by applying 2×500 microliters of deionized water, collecting pass through volume in the same vial. The eluate was mixed, and then an aliquot filtered through a 0.45 micrometer filter prior to HPLC analysis (25 mm, 0.45 micrometer filters were obtained from Gelman, Ann Arbor, Mich.). The HPLC system used had the following parameters: pump model G1312A, autosampler model G1313A, thermostatted column compartment model G1316A, diode array detector model G1315A, and peak integrator/data processor model G2170AA, all obtained from Agilent Technologies, Palo Alto, Calif. Column: 6.5×150 mm ION-310, 8 micrometer from Interaction Chromatography, San Jose, Calif. The system was pre-equilibrated in mobile phase (5 mN H₂SO₄) at 40° C. at 0.3 mL/min. prior to use.

For analysis a 10 microliter aliquot of sample or standard was injected, and the column was eluted with mobile phase at 0.3 mL/min. and 40° C. Eluting materials were detected by UV absorption at 210 nm and 220 nm. The run time was 45 min.

Unknown sample concentrations were determined by comparison to standards. Three aqueous solutions of pyroglutamic acid are usually sufficient as standards, i.e., 10, 20, and 40 mg/L (pyroglutamic acid obtained from Fluka, Milwaukee, Wis.).

N-acetyl-L-glutamine in the liquid nutritional type product showed no degradation during sterilization or after 3 months room temperature storage. Results are reported in Table 4. A small peak corresponding to N-acetyl-glutamic acid was detected at all time points, but remained at approximately the same level indicating no measurable degradation to N-acetyl-glutamic acid.

In the glutamine supplemented product, glutamine was reduced to about ⅓ the original concentration by the sterilization process; and by 2 months no glutamine was detected. In this product, pyroglutamic acid was detected at a concentration consistent with complete conversion of glutamine.

TABLE 4
Comparison of Stability of N-acetyl-L-glutamine and glutamine in
Liquid Nutritional Type Products During Processing
and over 3 Months of Storage at Room Temperature.

| | Product with | | |
|---|---|---|---|
| | N-acetyl-L- | | |
| | glutamine | | |
| | N-acetyl-L- | Product with glutamine | |
| | glutamine | Glutamine | pyroglutamic acid |
| Analyte | (mmol/L product) | (mmol/L product) | (mmol/L product) |
| Theoretical | 87.7 | 87.6 | — |
| Pre- | 92.5 | 97.8 | 27.1* |
| Sterilization | | | |
| Post- | 89.8 | 34.2 | 77.5* |
| Sterilization | | | |
| 1 month | 89.8 | 13.7 | 92.9* |
| 2 months | 94.1 | trace | 79.8** |
| 3 months | 89.3 | none detected | 80.6** |

*calculated with response factor from glutamine standard. Proper standard was not available until later in the experiment.
**calculated with response factor from pyroglutamic acid standard.

### Example 3

### Glutamine and N-Acetyl-L-Glutamine Bioavailability

Studies were conducted to determine the proportion of bioavailable N-acetyl-L-glutamine in comparison to glutamine in pig models. The intestinal loop model employs a section of isolated intestine to evaluate the absorption and metabolism of N-acetyl-L-glutamine and glutamine. The feeding model evaluated the absorption of N-acetyl-L-glutamine and glutamine when fed in a typical diet.

### Intestinal Loop Model

Twenty-two domestic pigs weighing 15-20 kg were acclimated to lab conditions over 4 days. The pigs were fed a standard pig diet, which followed energetic requirements for these animals (Nutrient Requirements of Swine, 9^{th}, 1998, Subcommittee on Swine Nutrition, National Research Council) and water ad libitum. Animals were randomly assigned into group C (6 pigs, receiving a glucosaline solution (Braun cat No 622647), 5% glucose, 0.9% NaCl), group G (8 pigs, receiving the same glucosaline solution fortified with 8 g/l of Gln, Sigma cat No G-3126), and group N (8 pigs, receiving the same glucosaline solution fortified with 10 g/l of NAQ, Sigma cat No A-9125). Before surgery, animals were fasted 15 h. The day of experiment, animals were weighed and anaesthetized using Stresnil^{R} and penthotal. The anaesthetized pigs were opened by abdominal medium sagital incision. Approximately 1 meter of proximal jejunum, about 1 meter from the ligament of Treitz, was, after clamping both ends and inserting a proximal fistual, filled with 125 mL of study solution at 50-75 mL/min. Intestinal infused solution samples were taken by puncture of infused intestine at 0, 15, 30, 60, 90, 120, 150 and 180 minutes. Samples were frozen in liquid nitrogen and maintained at −80° C. until analysis. Portal vein blood samples were taken by portal vein puncture at 0, 15, 30, 60, 90, 120, 150 and 180 minutes in tubes with anticoagulant. Samples were maintained at 4° C. until centriftigation at 1500×g for 15 minutes for plasma and red blood cell separation. Plasma was frozen at −20° C. until analysis. Jugular vein blood samples were taken by puncture at 0, 60, 120 and 180 minutes in tubes with anticoagulant and plasma obtained and stored as for portal blood vein. After 3 hours, pigs were sacrificed and mucosa samples were obtained from 25 cm of infused intestine segment. The segment was rinsed thoroughly with ice-cold saline solution, opened lengthwise and blotted dry. Mucosa were removed by scraping the entire luminal surface with a glass coverslip, then frozen in liquid nitrogen and stored at −80° C.

The analysis for N-acetyl-L-glutamine was conducted as follows. For intestinal infused solution samples and plasma samples, aliquots were diluted 1:10 (w/v) with 0.05% perchloroacetic acid (PCA) solution in water. For mucosa samples, 0.2 mg of wet mucosa sample was homogenized with 5 mL of 0.05% PCA solution in water. After centrifugation (15,000×g, 3 minutes, ambient temperature), samples were filtered through 0.45 micrometer filter and injected into an HPLC chromatographic system consisting of a 2690 Separation Module, PDA detector and a LichroCart 250-4 cartridge (Purospher RP18 e, 250×4 mm, 5 micrometers). The mobile phase consisted of a phosphate buffer 0.1 M at pH 2.7, at a flow rate of 1 mL/minute. The detection and quantification of N-acetyl-L-glutamine was monitored at 210 nm.

The analysis for glutamine and glutamate was conducted as follows. Intestinal infused solution samples and plasma samples were prepared as for N-acetyl-L-glutamine analysis (described above) with the exception that samples were diluted 1:400 (w/v) with 0.05% PCA solution in water. After samples were filtered through 0.45 micrometer filter. 20 microliters of the mixture was derivatized following the AccQ-Tag method (Waters Corp.), and diluted to 1 mL with water. Briefly, the sample was buffered with a borate solution and derivatized with 20 microliters of reactive. After 1 minute the sample was diluted to 1 mL and injected into the HPLC system, consisting of a 2690 Separation Module, fluorescence detector and a SupelcoSil LC-18 column (250×4 mm, 3 micrometers). Mobile phase consisted of a phosphonate buffer 0.1 M at pH 7.5, with 0.25% triethylamine and 9% acetonitrile, at a flow rate of 1 mL/minute. The detection and quantification of glutamate and glutamine was accomplished using an excitation wavelength of 250 nm and monitoring emission at 395 nm.

Glucose was analyzed using a well-established coupled enzyme assay. Briefly, sample glucose is phosphorylated using hexokinase and ATP (adenosine triphosphate), and the resulting glucose-6-phosphate is converted to 6-phosphogluconate using glucose-6-phosphate dehydrogenase. During the later reaction, NAD (nicotinamide adenine dinucleotide) is converted to NADH (the reduced form of NAD), resulting in increased absorbance at 340 nm, which is proportional to the glucose concentration in the original sample. This assay can be purchased as a clinical chemistry kit from Sigma Chemical Company, St. Louis, Mo., (current catalog number 16-20).

### Results

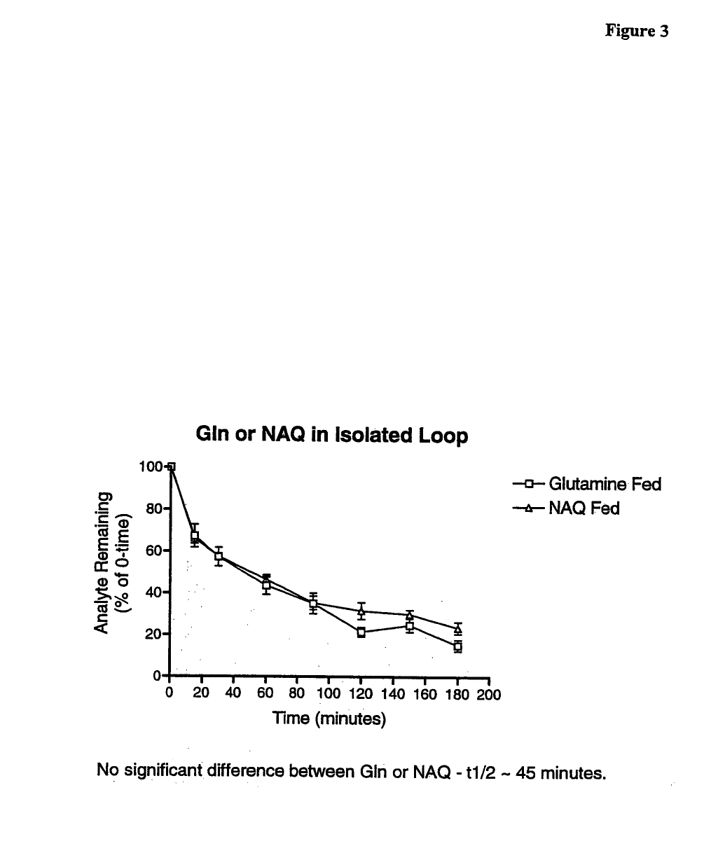

Glutamine or N-acetyl-L-glutamine remaining in the intestinal lumen versus time after introduction of the infused solution. The remaining percentage of glutamine or N-acetyl-L-glutamine in intestinal contents of pigs infused with solutions containing equivalent amounts of glutamine or N-acetyl-L-glutamine was similar during the first 90 minutes. There were statistically significant differences between groups at 120 and 180 minutes. There were no significant differences between glutamine or N-acetyl-L-glutamine at t_{1/2} (approximately 45 minutes). FIG. 3 illustrates graphically the amount of analyte (glutamine or N-acetyl-L-glutamine) remaining in the intestinal lumen versus time after introduction of the analyte. The analyte remaining is expressed as a percentage of the analyte present at time zero.

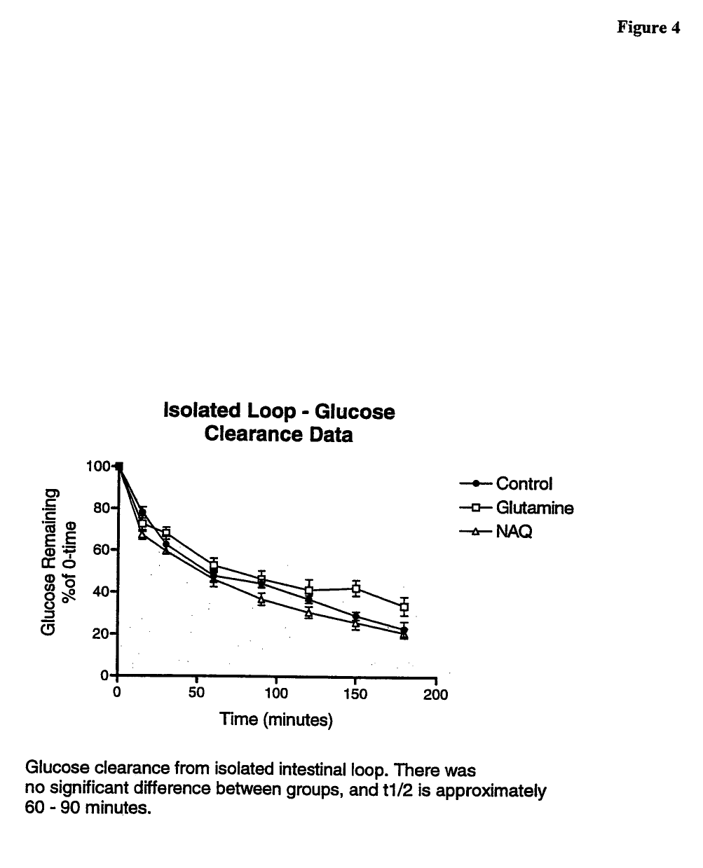

Glucose remaining in the intestinal lumen versus time after introduction of the infused solution There were no significant differences between C and G groups at any time. There were no significant differences between the C and N except at 15 minutes. G and N groups tended to be different from time 120 minutes, although penalizing by the Bonferroni's correction the only significant difference was at 180 minutes. FIG. 4 illustrates graphically the amount of glucose remaining in the intestinal lumen versus time after introduction of the solutions. Glucose remaining is expressed as a percentage of the amount present at time zero.

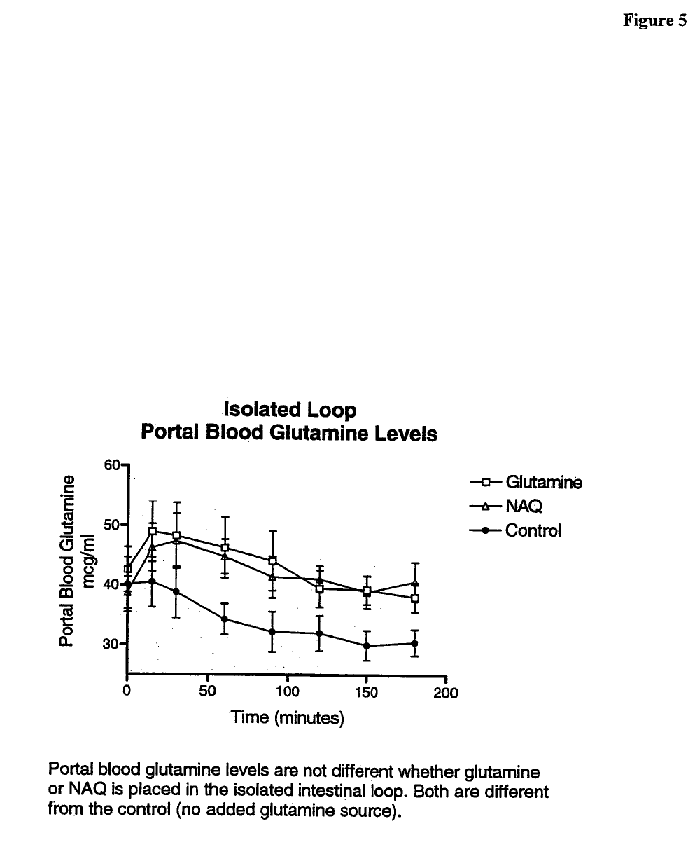

Glutamine in portal blood after introduction of the test solution into the intestinal loop. When results were expressed as percentages of the initial concentration, there were significant differences between control (C) and glutamine (G) and between C and N-acetyl-L-glutamine (N) groups (at 90 and 150 minutes, C vs. G; and at 90, 120, 150 and 180 minutes, C vs. N). There were no significant differences between G and N. When results were expressed as absolute values, there were no significant differences between groups except at 120 minutes, between C and N. Taken together, G and N tend to be different from C from 120 minutes to the end of the experiment. FIG. 5 illustrates graphically the amount of glutamine in the portal blood (in mcg/mL) versus time after introduction of the test solution into the intestinal loop.

There were no significant differences between groups for glucose in portal blood and between groups for glutamine or glucose in peripheral blood. There were only negligable (parts-per-million) levels of intact N-acetyl-L-glutamine detected in either portal or peripheral blood at any time point during the experiment.

### Glutamic Acid (GLU) and Glutamine (GLN) in Jejunum Mucosa

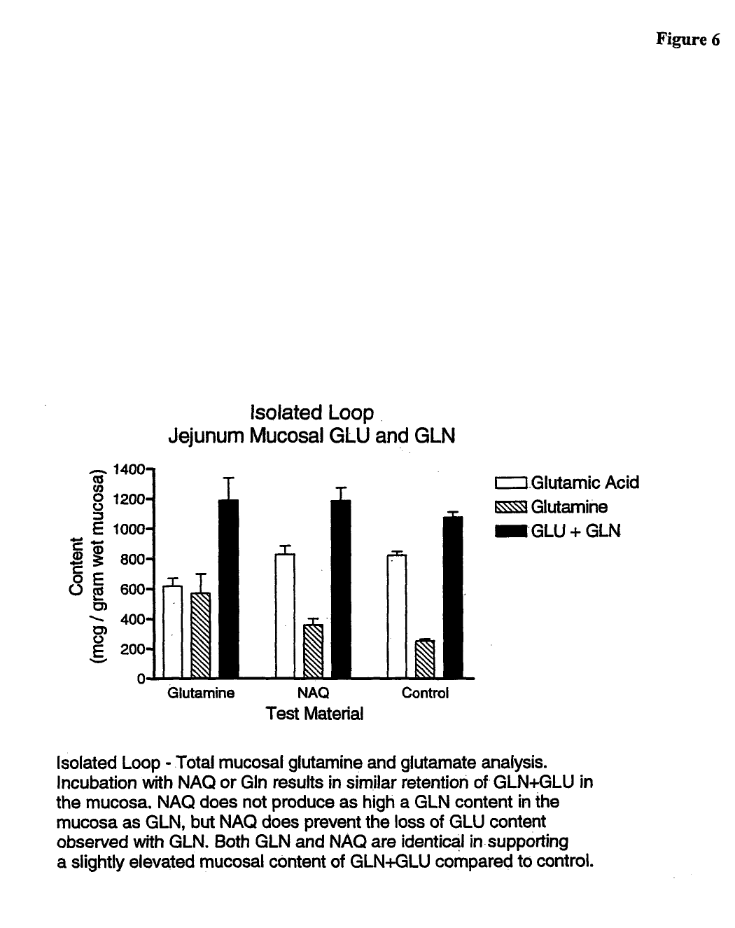

There were higher glutamate concentrations in groups N and C than in group G, and, while both N and G groups showed higher glutamine in the mucosa, group G was substantially higher than group N. However, the sum glutamine+glutamate concentration were similar in groups G and N, suggesting that delivery of glutamine carbon skeleton to mucosal metabolic systems is comparable using these two diets. Intact N-acetyl-L-glutamine could not be detected in mucosa samples. FIG. 6 illustrates graphically the amount of glutamine and glutamate (and their sum) in the jejunum mucosa immediately following completion of the experiment (expressed in mcg/gram wet mucosa).

In summary, N-acetyl-L-glutamine shows a similar bioavailability to glucose and very slightly lower than glutamine. N-acetyl-L-glutamine seems to be very similar to glutamine in utilization after absorption. After being absorbed, N-acetyl-L-glutamine is quickly hydrolyzed by enterocyte acylase, entering in the normal glutarnine metabolism, and achieving glutamine+glutamate concentration in mucosa as high as that achieved by an equivalent glutamine diet. Excess glutamine is excreted to the portal vein, where glutamine concentration is similar to that found after an equivalent dose of dietary glutamine. N-acetyl-L-glutamine concentration in portal vein plasma is only a few ppm, suggesting minimal intact absorption to the bloodstream. The high rate of absorption of N-acetyl-L-glutamine as well as a similar metabolism to glutamine suggested that both nutrients could have the same biological behavior under catabolic stages of the organism.

### Feeding Pig Model

Fifteen pigs, 15-20 kg in weight were provided by a certified farm. The pigs were acclimated to the laboratory for 2 days. A standard pig diet and water was provided ad libitum. After acclimation, the pigs were randomly assigned into group C (5 pigs, receiving a standard pig diet plus 3 g/kg of Cr₂O₃, Merck cat No 1.02483), group G (5 pigs, receiving diet C plus 8 g/kg of Gln, Ajimoto), and group N (5 pigs, receiving diet C plus 10.5 g/kg of N-acetyl-L-glutamine, Flamma). During the experimental phase of the study, each group received 1000 grams of their respective diet per day per animal, fed in 3 portions and water was provided ad libitum. This experimental phase of feeding lasted 5 days.

On the day of experiment, animals were weighed and received the standard diet intake (333 g diet per animal) at 7:00 a.m. Three hours after feeding, animals were weighed, sedated and bled through jugular vein puncture. Animals were quickly opened by abdominal medium sagital incision and the content of the duodenum, medium jejunum (about 2 meters from the ligament of Treitz) and ileum (30 cm from the ileocecal valve) were taken, frozen in liquid nitrogen, lyophylized, and stored at −80° C. until analysis. Samples of liver and kidney were removed, dissected of visible fat and connective tissue, quickly frozed in liquid nitriogen and stored at −80° C. until analysis. Samples of intestinal mucosa were obtained as described for the isolated intestinal loop experiment, and stored as described above prior to analysis.

Intestinal content was analyzed for glutamine, N-acetyl-L-glutamine and chromium (III) oxide. For analysis of N-acetyl-L-glutamine, the lyophilized samples of intestinal content were dissolved 1:20 (w/v) with 0.05% PCA in water followed by HPLC analysis as described in the Intestinal Loop model above.

For analysis of glutamine, the lyophilized intestinal content was treated and analyzed as described in the intestinal loop model above.

Chromium was incorporated into the diets to provide a correction factor to reflect content per kg of original diet. For analysis of chromium (III) oxide the following procedure was utilized. A representative lyophilized intestinal content sample was weighed into a nickel crucible and placed in a muffle furnace. Temperature was raised to 500° C. and maintained for a further 2 hours. After cooling, a fusion mixture (Na₂CO₃ K₂CO₃ KNO₃, 10:10:4 w/w/w) was added at about ten times the weight of sample ash and mixed thoroughly. An extra amount of fusion mixture was added to form a thin layer on top and fused for 30 minutes over an open flame using a gas burner until a clear melt was obtained. The crucible was removed from the burner, allowed to cool, and the melt was extracted thoroughly by washing the walls with about 20 mL of water and then heated gently on the hot plate for about 30 minutes. When the crust was thoroughly loosened, the crucible was rinsed four times with water, and all washings were added to a 100 mL volumetric flask water, and diluted to volume. The absorbance at 372 nm against demineralized water as a blank was determined. The absorbance readings were converted to mg of Cr₂O₃ by employing the equation of a standard curve prepared by analyzing 0, 50, 100, 200 and 500 microliters of a standard chromium solution (2.9034 g of K₂Cr₂O₇/L, which is equivalent to 1.5 g/L of Cr₂O₃).

Analysis for acylase was conducted according to the following procedure. 200 mg of wet mucosa, liver or kidney was homogenized into 5 mL of cold water and centrifuged at 400×g for 5 minutes at 5° C. 100 microliters of an N-acetyl-L-glutamine solution (5 g/L, sigma catalog no. A-9125), were mixed with 100 microliters of mucosa homogenate and incubated during 1 hour at 37° C. A blank was done using 100 microliters of mucosa and 100 microliters of water. An enzyme calibration curve was constructed (acylase I, E.C. 3.5.1.14, Sigma catalog no. 8376), using from 0.5 IU acylase /mL to 100 IU acylase/mL, and incubating with N-acetyl-L-glutamine as above. Free glutamine (released by enzyme activity) was determined as described the intestinal loop model above. For each sample, the acylase activity was determined by comparison to the standard response curve for the enzyme, and the value corrected by appropriate dilution factors.

### Results

Absorption data are presented in Table 5 below. Samples from the duodenum contained insufficient levels of chromium (II) oxide to allow quantitation. The analytical results could not be corrected to reflect content per kg of original diet. The medial jejunum contained essentially identical levels of glutamine (in the case of diet G) and N-acetyl-L-glutamine (in the case of diet N), suggesting similar adsorption in the duodenum and proximal jejunum. However, these diets also contained intact protein, and digestion of that protein could also produce significant free glutamine, as indicated by the analysis result for the control diet. This suggests that the free glutamine content of the original diet is almost completely absorbed prior to the medial jejunum. Analysis of the contents of the distal ilium suggest that, while absorption of free glutamine can continue between the medial jejunum and the distal ilium, absorption of N-acetyl-glutamine is not observed. However, overall absorption data indicate absorption of approximately 77% of the high level of administered N-acetyl-L-glutamine in this model.

TABLE 5
Adsorption of N-acetyl-L-glutamine
and Glutamine as a Component of Diet in Pigs.

| | | N-acetyl- | |
|---|---|---|---|
| | Glutamine Diet | L-glutamine Diet | Control Diet (C) |
| Duodenum | N/D** | N/D | N/D |
| Medium Jejunum | 10.1 ± 1.9 | 10.3 ± 2.4 | 8.8 ± 0.7 |
| Distal Ileum | 1.2 ± 0.6 | 12.8 ± 2.1 | 2.1 ± 0.7 |

*For Glutamine and Control diets, data are glutamine (mmole/kg original diet). For N-acetyl-L-glutamine diet, data are for N-acetyl-L-glutamine (mmole/kg original diet). Original diets are (Glutamine = 54.8 mmole glutamine/kg diet, N-acetyl-L-glutamine = 55.8 mmole N-acetyl-L-glutamine/kg diet).
**N/D = Not determined. Chromium (II) oxide values were below quantitation limits for the assay, and corrected values could not be generated.

Acylase activity in intestinal mucosa, liver and kidney—Acylase activity was measured in several tissues of interest (in view of likely nutritional importance) in the control pigs. Acylase activity was found in all tissues tested, including jejunal mucosa, liver and kidney. Levels determined were 948±300 IU/g wet tissue (17.3±7.0 IU/mg protein) in the jejunal mucosa, 12,770±1110 IU/g wet tissue (159±30 IU/mg protein) in liver and 19,630±3020 IU/g wet tissue (302±47 IU/mg protein) in the kidney.

In summary, N-acetyl-L-glutamine was absorbed mainly in the duodenum and upper-jejunum, where at least 77% of the dose was adsorbed. There were two main differences between N-acetyl-L-glutamine and glutamine: an earlier N-acetyl-L-glutamine uptake saturation and a lower ileal absorption.

### Example 4

### Effects of N-Acetyl-L-Glutamine on Intestinal Damage Caused by Malnutrition

A study was conducted to evaluate the potential effects of N-acetyl-L-glutamine versus free glutamine on intestinal damage caused by protein-energy malnutrition in pigs. In this study, 24 domestic pigs, 5 weeks old, were provided by a certified farm. The pigs were randomly assigned to one of two groups. In one group 6 pigs were freely fed with ENSURE PLUS® (Ross Products Division, Abbott Laboratories) for 30 days. In the second group, 18 pigs were also fed with ENSURE PLUS®, but at only 20% of the daily intake of the first group. This second group was divided into 3 subgroups with six pigs each to receive a daily supplement of either calcium caseinate, glutamine or N-acetyl-L-glutamine. Daily average energy and protein supplied to the control group ranged from 3300 kcal, 138 g protein at the beginning of the study to 4500 kcal, 187 g protein at the end of the study. In the second group, supplements of caseinate, glutamine and N-acetyl-L-glutamine provided an additional 1.32 grams nitrogen equivalents per day (basically, 6.89 grams L-glutamine, or 8.87 grams N-acetyl-L-glutamine or 8.42 grams caseinate protein are supplemented per day). After 30 days, all pigs were deprived of food for 16 hours. The animals were then weighed, sedated, anesthetized and sacrificed through terminal bleeding by jugular puncture.

The entire small intestine was quickly removed. A 5 cm segment of the small intestine from the ligament of Treitz was selected for histological analysis. The next 60 cm was considered the proximal jejunum for biochemical measurements. The 60 cm length closest to the ileo-cecal valve was considered the distal ileum. The intestine segments were rinsed thoroughly with ice-cold saline solution, opened length-wise and blotted dry. The mucosa was scraped off using a glass slide onto a cold Petri dish, weighed, immediately frozen under liquid nitrogen and stored at −80° C. until biochemical analysis.

Jejunal and ileal mucosa were homogenized in 10 mM phosphate buffer (pH 7.4) using a mechanical Potter homogenizer, for protein and DNA assays. For the determination of the enzymatic markers of injury, functionality and antioxidant defense system, the mucosal homogenates were centrifuged at 3000 g for 10 min. and the resulting supernatants were used for enzymatic assays. For the determination of total glutathione, the mucosa was homogenized in 5% trichloroacetic acid and centrifuged at 8000 g for 5 min.

Biochemical analysis and immunological analysis were performed on the specimens. Concentrations of intestinal mucosa protein and DNA were determined using the Bradford method (Analytical Biochemistry, Volume 72, pages 248-254, 1976) and the method of Labarca and Paigen (Analytical Biochemistry, Volume 102 (2), pages 344 - 352, 1980), respectively. The degree of intestinal damage caused by malnutrition was evaluated by measuring alkaline phosphatase activity using the method of Goldstein (R. Goldstein, T. Klein, S. Freier and J. Menczel. American Journal of Clinical Nutrition 24: 1224-1231, 1970).

The defensive system against oxidative damage was evaluated by measuring the activities of glutathione reductase (GR), glutathione transferase (GT) and glutathione peroxidase (GPOX) as well as by the concentration of the non-protein sulflhydryl groups (mostly reduced glutathione (GSH)). Glutathione reductase activity was evaluated by the method of Carlberg and Mannervik (I. Carlberg and B. Mannervik, Methods in Enzymology, Volume 113, pp 484-490, 1985). Glutathione transferase activity was measured using the method of Habig, et al. (W. H. Habig, M. J. Pabst and W. B. Jakoby, Journal of Biological Chemistry. 294: 7130-7139, 1984). Glutathione peroxidase activity was assayed by the method of Flohe and Gunzler (L. Flohe and W. A. Gunzler, Methods in Enzymology, Volume 105, pp 114-121, 1984), and the non-protein sulfhydryl content (reported as reduced glutathione equivalents) was determined by the method of Anderson (M. E. Anderson, Methods in Enzymology, 133: 548-554, 1985).

Intestinal lymphocytes were isolated following the procedure of Gautreaux, et al. (M. D. Gautreaux, E. A. Deitch and R. D. Berg, Infection and Immunity 62(7): 2874-2884, 1994) modified as detailed below. Two small intestine segments from jejunum and from ileum respectively, were isolated and the luminal content was flushed with phosphate-buffer saline (PBS, Sigma St. Louis, Mo., USA). The visible Peyer's patches were excised, and the intestine was opened longitudinally and cut into small pieces. To isolate the small intestinal epithelium those pieces were incubated for 30 min at 37° C. in 25 ml of Hanks Balanced Salt Solution (HBSS; Sigma, St. Louis, Mo., USA) with 5 mM dithiotreitol (DTT; Roche Molecular Biochemicals, Indianapolis, Ind., USA), 2mM EDTA (Sigma, St. Louis, Mo., USA) and 25 mM Tris buffer (Sigma, St. Louis, Mo., USA) in a shaking water bath (120 strokes per min); the supernatant was decanted, fresh HBSS-DTT-EDTA-Tris was added, and the incubation procedure was repeated. The supernatants containing the epithelial cells from two incubations were pooled, and the cells were washed by centrifugation with rpmi 1640 culture medium containing 5% (v/v) heat-inactivated fetal calf serum (Sigma, St. Louis, Mo., USA), 20 mM HEPES (Sigma, St. Louis, Mo., USA), 2 mM L-glutamine, 500 U penicillin and 100 μg/ml streptomycin (Sigma, St. Louis, Mo., USA)(complete medium). Lamina propria lymphocytes (LPL) were liberated from the remaining sediment by placing the intestinal debris in 40 ml of complete medium with collagenase 0.05 U/ml, dispase 0.30 U/ml (Sigma, St. Louis, Mo., USA) and DNase I 500 U/ml (Roche Molecular Biochemicals, Indianapolis, Ind., USA) for 120 min in a 37° C. shaking water bath at 120 strokes per min. The excised Peyer's patches were placed in complete medium and dissected with a couple of scalpels. The cleaned Peyer's patches were then collagenase treated (reduced incubation time to 60 min.) as described above for LPL isolation to liberate Peyer's patch lymphocytes (PPL).

Each of the cell types isolated from the epithelium, the lamina propria and Peyer's patches were subjected to discontinuous Percoll (Sigma, St. Louis, Mo., USA) density gradient centrifugation to enrich for lymphocytes. The commercial Percoll solution was diluted 9:10 with 9% NaCl yielding an isotonic Percoll solution that was diluted with complete medium to obtain 3 solutions differing in percent Percoll concentration (75%, 40% and 30%), which were used in decreasing order. The cells were resuspended in 4 ml of complete medium and were placed over the 30% fraction. After centrifugation at 650 g for 20 min, the interfaces between the 75 and 40% layers were removed and the cells were washed by centrifugation in 25 ml of complete medium. The cells were then resuspended in 4 ml of 40% Percoll and centrifugated at 650 g. The cell pellets, enriched for lymphocytes (IEL, LPL and PPL), were collected and washed by centrifugation with PBS.

The isolated lymphocytes were stained with monoclonal antibodies quantitated by flow cytometry as follows: One hundred μl of each lymphocyte preparation (2×10⁶ cel/ml) were placed in 3-ml tubes with different concentration of monoclonal antibodies (Anti CD1 FITC, Anti CD3ε FITC, Anti CD4a PE, Anti CD8a PE, Anti CD11b/Mac-1 APC, Anti CD21 APC), and were incubated for 30 min. in dark at 4° C. The cells were washed with PBS, pelleted by centrifugation (500 g, 5 min.), and resuspended in 350 μl PBS.

Fluorescence-activated cell sorter (FACS) analysis of cell preparations was carried out on a FACScalibur flow cytometer (Becton Dickinson). Nonspecific fluorescence was determined through 3 controls (for fluorescein isothyocyanate—FITC, phycoerythrin—PE and allophycocyanin—APC) prepared for each cell preparation.

### Biochemical Results

Reduction of dietary intake to 20% of control resulted in a complete failure to grow. Malnourished pigs lost an average of 2-3 kg of total weight, while control pigs gained 18 kg during the 30 day trial. Liver weight and the weight per length of both jejunal and ileal mucosa were also severely reduced as consequence of malnutrition (Table 6).

TABLE 6
Liver and small intestinal weights of control
and protein-energy malnourished pigs.

| | | Weight Mucosa/Length Intestine (g/cm) | |
|---|---|---|---|
| | Liver Weight (g) | Jejunum | Ileum |
| Control Pigs | 731.4 ± 26.5 | 0.092 ± 0.008 | 0.070 ± 0.007 |
| Mal- | 237.9 ± 9.9* | 0.035 ± 0.005* | 0.025 ± 0.006* |
| nourished | | | |
| Pigs | | | |

*Significant difference vs. control group (p < 0.05).

The amounts of DNA and protein per length of mucosa were significantly lower (2 to 3 fold) in malnourished pigs compared with controls (data not shown). However, the protein/DNA ratio was not affected by PEM in any intestinal segment. These results suggest that the overall process of protein and DNA synthesis in the small intestine of malnourished pigs is impaired. The intestinal contents of protein (jejunum and ileum) and DNA (ileum) tended to be higher in the malnourished pigs that consumed NAQ supplement than in those that consumed caseinate or glutamine. These results suggest that NAQ partially preserves the protein and DNA synthesis process during the malnutrition period.

Alkaline phosphatase segmental activity, as marker of intestinal injury, was significantly lower (2 to 3 fold) in malnourished pigs than in controls in jejunal segment (data not shown). In the ileal segment, alkaline phosphatase activity was less affected by the malnutrition process. In addition, malnourished pigs that consumed the glutamine or NAQ supplements tended to have higher AP activity in jejunum than those that consumed caseinate supplement.

Glutathione is the central component of the whole antioxidant defense system. It is an effective free radical scavenger and is also involved in a range of other metabolic functions, including the maintenance of protein sulfhydryl groups in the reduced state, cofactor for GT and GPX, amino acid transport, and protein and DNA synthesis. The total glutathione concentration was significantly reduced in both small intestinal segments of the malnourished pigs in comparison to the control group. However, the amount of GSH in the intestinal mucosa of malnourished pigs that consumed NAQ tended to be slightly higher than in those that consumed the caseinate or glutamine supplements, though this difference did not reach significance.

Glutathione transferase and glutathione reductase enzymatic activities, responsible of aldehyde detoxification and of glutathione reduction, respectively, were found reduced (again, 2 to 3 fold) in the small intestine as a consequence of malnutrition. Depression in the glutathione transferase activity could aggravate the intestinal dysfunction by accumulation of aldehydes, epoxides and other products containing electrophilic centers within the mucosa. This activity looked to be less affected by the malnutrition process in the pigs that consumed the N-acetyl-L-glutamine supplement. The activity of glutathione reductase and of glutathione peroxidase were also reduced 2 to 3 fold by malnutrition in both small intestinal segments. Glutathione reductase is involved in glutathione regeneration from its oxidized form, and glutathione peroxidase oxidizes two reduced glutathione molecules to detoxify peroxides. A tendency of reduced glutathione to be higher in the intestinal mucosa of pigs fed with the N-acetyl-L-glutamine supplement was associated with a tendency of glutathione peroxidase activity to be higher in the same group.

In summary, the deleterious effects of malnutrition on the antioxidant defense system appeared less marked in the intestine of animals that consumed the N-acetyl-L-glutamine supplement than in the animals that consumed the caseinate or glutamine supplements.

### Immunological Results

There was a decrease in the total number of small intestine peyer's patch lymphocytes as a result of malnutrition. In ileum, the total number of peyer's patch lymphocytes was significantly lower in caseinate- and glutamine-supplemented pigs than in the N-acetyl-glutamine-supplemented or the control groups. In jejunum, there was also a tendency of the total number of peyer's patch lymphocytes to be higher in N-acetyl-L-glutamine- than in caseinate- or glutamine-supplemented groups. On the other hand, the total number of jejunum intra-epithelial lymphocytes was significantly higher in all malnourished groups compared to the control group. No differences were found in the number of lymphocytes in the lamina proprial of small intestine for any experimental group.

In all malnourished groups the number of peyer's patch lymphocytes expressing B cell markers (CD1 and CD21) were lower than in healthy group, being especially significant in the case of CD1+lymphocytes. The reduction in the number of CD21+peyer's patch lymphocytes compared to control group in ileum was significantly different in the caseinate- and glutamine-supplemented groups, but not in N-acetyl-L-glutamine supplemented group. In jejunum, there was the same tendency but did not reach statistical significance. The reduction in the number of CD11b+peyer's patch lymphocytes in jejunum and ileum also showed a tendency to be lower in N-acetyl-L-glutamine-supplemented than in caseinate- or glutamine-supplemented groups.

The number of T cells (CD3+cells) in jejunum and ileum peyer's patch lymphocytes decreased with malnutrition. The decrease was due to both helper (CD4+) and citotoxic (CD8+) T cells. However, there was a general tendency of this decrease of T cells in PPL to be lower in the N-acetyl-L-glutamine-supplemented than in the caseinate- or glutamine-supplemented groups. In some cases, such as in CD4+ and CD8+cells in ileum, significant differences were detected between control and caseinate- or glutamine-supplemented groups, but not between the control and N-acetyl-L-glutamine-supplemented groups.

As noted above, malnutrition promoted an increase in the total number of intra-epithelial lymphocytes in jejunum. This increase was detected in both populations, B cells (CD21+) and T cells (CD3+). In B cells, the number of CD1+lymphocytes in the N-acetyl-L-glutamine supplemented group was significantly higher than in the rest of the groups. In T cells, T cytotoxic subpopulations (CD8+) was significantly higher in all the malnourished groups than in the control group. However, the T helper (CD4+) subpopulation was significantly higher in glutamine- and N-acetyl-L-glutamine-supplemented groups (but not in caseinate-supplemented group) than in the control group. This indicates a selective effect of glutamine and N-acetyl-L-glutamine on the T helper (CD4+) subpopulation. No significant differences were detected for any of the lymphocyte subpopulations in ileum intra-epithelial lymphocytes.

There were no substantial important changes in lamina propria lymphocytes due to malnutrition. There was a reduction of the number of CD2 1+cells (B cells) in the caseinate-supplemented group compared to the control group that was not detected in either the glutamineor N-acetyl-L-glutamine-supplemented groups. In addition, the N-acetyl-L-glutamine-supplemented group, but not the glutamine-supplemented group was significantly different from the caseinate-supplemented group.

In summary, the N-acetyl-L-glutamine-supplemented group performed better than the glutamine or caseinate supplemented groups, showing statistically significant differences, to reduce small intestine immunological changes promoted by malnutrition, especially in total cell number and B and T helper subpopulations.

### Conclusions

Under normal physiological conditions, there is a steady state balance between the production of oxygen-derived free radicals and their destruction by the cellular antioxidant system. In the present study, the intestinal balance was upset by protein-energy malnutrition, leading to a decrease in reduced glutathione and in the enzymatic antioxidant defense system. In addition, intestinal immune response was severely impaired by protein-energy malnutrition.

Although no clear effect of glutamine was detected on the prevention of biochemical and immunological changes induced by malnutrition in the small intestine, probably due to the fact that malnutrition was especially severe, there was a positive effect of N-acetyl-L-glutamine to reduce the severity of these changes.

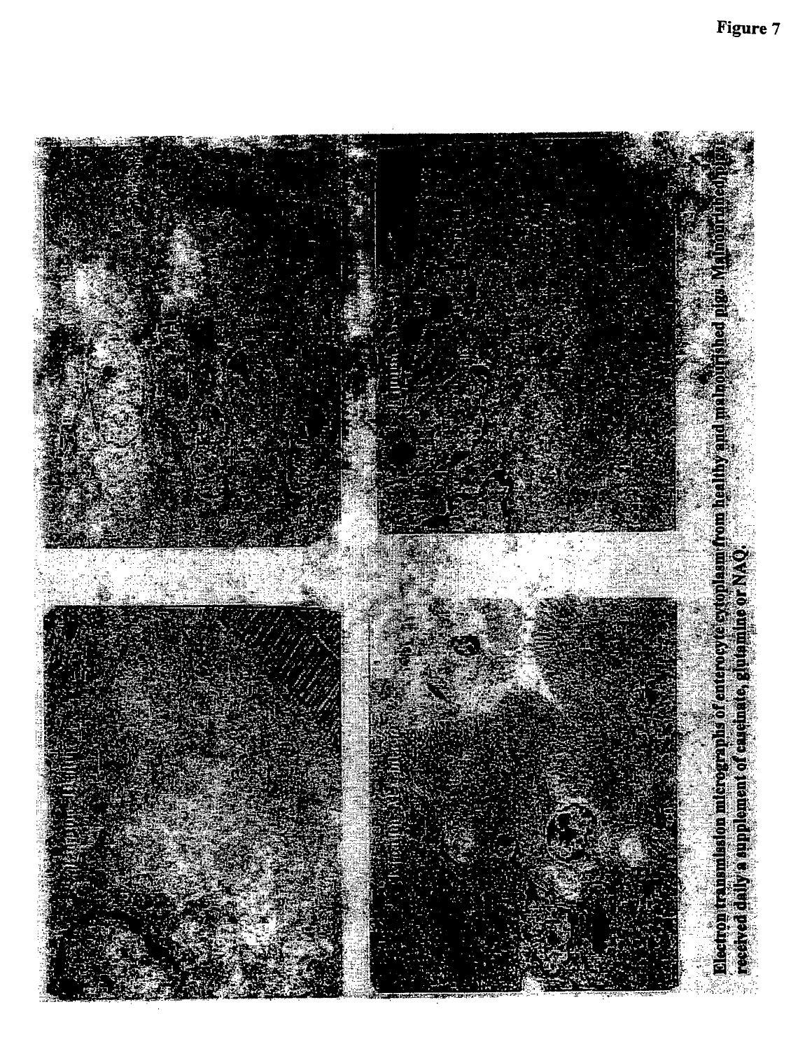

This study suggests that N-acetyl-L-glutamine has a positive effect on the cells of the small intestine, even beyond that of glutamine. Additionally, electron transmission micrographs of enterocyte cytoplasm from healthy and malnourished pigs are shown in FIG. 7. These micrographs shows that N-acetyl-L-glutamine is more effective than glutamine at preventing the overt signs of inflammation in the epithelial lining of the gastrointestinal tract.

Particular embodiments have been described above that fall within the scope of the invention as set forth in the claims. These embodiments are not intended to limit the scope of the invention to the specific forms disclosed. The invention is intended to cover all modifications and alternative forms falling within the spirit and scope of the invention.

What is claimed is:

1. An aqueous solution containing:
a) from 30 mEq to 95 mEq of sodium per liter;
b) from 10 mEq to 30 mEq of potassium per liter;
c) from 10 mEq to 40 mEq of citrate per liter;
d) less than 3.0 wt./wt. % of one carbohydrate; and
e) at least 5.0 mmoles N-acetyl-L-glutamine, or a nutritionally equivalent salt thereof, per liter of solution.

2. An aqueous solution according to claim 7, containing 20 mmoles to 300 mmoles of N-acetyl-L-glutamine or a nutritionally equivalent salt thereof per liter of solution.

3. An aqueous solution according to claim 7, containing 25 mmoles to 200 mmoles of N-acetyl-L-glutamine or a nutritionally equivalent salt thereof per liter of solution.

4. An aqueous solution according to claim 7, wherein said nutritionally acceptable salt is selected from the group consisting of: lithium, sodium, potassium, calcium, magnesium, and aluminum, ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, N,N′-dibenzylethylenediamine, ethylenediamine, ethanolamine, diethanolamine, piperidine, piperazine, and mixtures thereof.

5. An aqueous solution according to claim 7, wherein said aqueous solution further contains chloride.

6. An aqueous solution according to claim 7, wherein said carbohydrate is a mixture of dextrose and fructose.

7. An aqueous solution according to claim 7, wherein said carbohydrate is present in a quantity of less than 3.0 wt/wt %.

8. An aqueous solution according to claim 7, wherein said sodium is present in the quantity of 30 mEq/L to 95 mEq/L.

9. An aqueous solution according to claim 7, wherein said sodium is selected from the group consisting of sodium chloride, sodium citrate, sodium bicarbonate, sodium carbonate, sodium hydroxide and mixtures thereof.

10. An aqueous solution according to claim 7, wherein said potassium is present in the quantity of 10 mEq/L to 30 mEq/L.

11. An aqueous solution according to claim 7, wherein said potassium is selected from the group consisting of potassium citrate, potassium chloride, potassium bicarbonate, potassium carbonate, potassium hydroxide and mixtures thereof.

12. An aqueous solution according to claim 11, wherein said chloride is present in the quantity of 30 mEq/L to 80 mEq/L.

13. An aqueous solution according to claim 11, wherein said chloride is selected from the group consisting of potassium chloride, sodium chloride, and zinc chloride.

14. An aqueous solution according to claim 7, wherein said citrate is present in the quantity of 20 mEq/L to 40 mEq/L.

15. An aqueous according to claim 7, wherein said citrate is selected from the group consisting of potassium citrate, sodium citrate, and citric acid.

16. An aqueous solution according to claim 7, further comprising at least one flavor.

17. An aqueous solution according to claim 7, further comprising at least one artificial sweetener.

18. An aqueous solution according to claim 7, further comprising at least one gelling agent selected from the group consisting of agar, alginic acid and salts, gum arabic, gum acacia, gum talha, cellulose derivatives, curdlan, fermentation gums, furcellaran, gelatin, gellan gum, gum ghatti, guar gum, iota carrageenan, irish moss, kappa carrageenan, konjac flour, gum karaya, lambda carrageenan, larch gum/arabinogalactan, locust bean gum, pectin, tamarind seed gum, tara gum, gum tragacanth, native and modified starch, xanthan gum, in a quantity sufficent to support a self supporting three dimensional structure.

19. An aqueous solution according to claim 7, further comprising rice flour.

20. An aqueous solution according to claim 7, further comprising an indigestible oligosaccharide.

21. A liquid nutritional formula comprising:
a) a protein component, which comprises from 8 to 35% of the total caloric content of said liquid nutritional formula;
b) a carbohydrate component, which comprises from 36 to 76% of the total caloric content of said liquid nutritional formula;
c) a lipid component, which comprises from 6 to 51% of the total caloric content of said liquid nutritional formula; and
to 23% on a caloric basis of the protein component in the form of N-acetyl-L-glutamine or a nutritionally acceptable salt thereof.

22. An adult liquid nutritional formula comprising:
a) a protein component, which comprises from 14 to 35% of the total caloric content of said liquid nutritional formula;
b) a carbohydrate component, which comprises from 36 to 76% of the total caloric content of said liquid nutritional formula;
c) a lipid component, which comprises from 6 to 51% of the total caloric content of said liquid nutritional formula; and
at least 35 mmoles of N-acetyl L-glutamine, or a nutritionally acceptable salt thereof, per 1000 kcal of nutritional formula.

23. A nutritional formula as defined in claim 28, wherein said formula comprises 35 mmoles to 160 mmoles of N-acetyl-L-glutamine, or a nutritionally acceptable salt thereof, per 1000 kcal of nutritional formula.

24. A liquid nutritional formula for a non-adult patient comprising:
a) a protein component, which comprises from 8 to 25% of the total caloric content of said liquid nutritional formula;
b) a carbohydrate component, which comprises from 39 to 44% of the total caloric content of said liquid nutritional formula;
c) a lipid component, which comprises from 45 to 51% of the total caloric content of said liquid nutritional formula; and
at least 5.0 mmoles of N-acetyl L-glutamine, or a nutritionally acceptable salt thereof, per 1000 kcal of nutritional formula.

25. A nutritional formula as defined in claim 30, wherein said formula comprises 5.0 mmoles to 32 mmoles of N-acetyl-L-glutamine, or a nutritionally acceptable salt thereof, per 1000 kcal of nutritional formula.

26. A liquid nutritional formula according to claim 27, wherein said nutritionally acceptable salt is selected from the group consisting of: lithium, sodium, potassium, calcium, magnesium, and aluminum, ammonium, tetramethylammonium, tetraethylammonium, methylamine, dimethylamine, trimethylamine, triethylamine, diethylamine, ethylamine, tributylaamine, pyridine, N,N-dimethylaniline, N-methylpiperidine, N-methylmorpholine, dicyclohexylamine, procaine, dibenzylamine, N,N-dibenzylphenethylamine, 1-ephenamine, N,N′-dibenzylethylenediamine, ethylenediamine, ethanolamine, diethanolamine, piperidine, piperazine, and mixtures thereof.

27. A liquid nutritional formula according to claim 27, containing less than 1.0 g of pyroglutamic acid per 1500 kcal of formula.

28. A liquid nutritional formula according to claim 27, wherein said formula is an adult formula, and the protein component comprises from 14 to 35% of the total caloric content of said liquid nutritional formula; the carbohydrate component comprises from 36 to 76% of the total caloric content of said liquid nutritional formula; the lipid component comprises from 6 to 41% of the total caloric content of said liquid nutritional formula.; and the N-acetyl-L-glutamine or a nutritionally acceptable salt thereof, comprises 1 to 25% on a caloric basis of the protein calories.

29. A liquid nutritional formula according to claim 27, wherein the formula is for non-adults, and the protein component comprises from 8 to 25% of the total caloric content of said liquid nutritional formula; the carbohydrate component comprises from 39 to 44% of the total caloric content of said liquid nutritional formula; the lipid component comprises from 45 to 51 of the total caloric content of said liquid nutritional formula; and the N-acetyl-L-glutamine or a nutritionally acceptable salt thereof, comprises 1 to 12% on a caloric basis of the protein calories.

30. A liquid nutritional formula according to claim 27, wherein said liquid nutritional formula is administered orally.

31. A liquid nutritional formula according to claim 27, wherein said liquid nutritional formula is administered enterally.

32. A liquid nutritional formula according to claim 27, further comprising vitamins and minerals selected from the group consisting of calcium, phosphorus, sodium, chloride, magnesium, manganese, iron, copper, zinc, selenium, iodine, chromium, molybdenum, minositol, carnitine, taurine, Vitamins A, C, D, E, K and the B complex, and mixtures thereof.

33. A liquid nutritional formula according to claim 27, wherein said lipid component is selected from the group consisting of coconut oil, soy oil, corn oil, olive oil, safflower oil, high oleic safflower oil, MCT oil (medium chain triglycerides), sunflower oil, high oleic sunflower oil, palm oil, palm olein, canola oil, fish oil, palm kernel oil, menhaden oil, soybean oil, cottonseed oil, lecithin, lipid sources of arachidonic acid and docosahexaneoic acid, structured lipids, and mixtures thereof.

34. A liquid nutritional formula according to claim 27, wherein said protein component comprises intact protein selected from the group consisting of soy based protein, milk based protein, casein protein, whey protein, rice protein, beef collagen, pea protein, potato protein, and mixtures thereof.

35. A liquid nutritional formula according to claim 27, wherein said protein component comprises hydrolyzed protein selected from the group consisting of soy protein hydrolysate, casein protein hydrolysate, whey protein hydrolysate, rice protein hydrolysate, potato protein hydrolsate, fish protein hydrolysate, egg albumen hydrolysate, gelatin protein hydrolysate, a combination of animal and vegetable protein hydrolysates, and mixtures thereof.

36. A liquid nutritional formula according to claim 27, wherein said protein component comprises free amino acids selected from the group consisting of tryptophan, tyrosine, cyst(e)ine, methionine, arginine, leucine, valine, lysine, phenylalanine, isoleucine, threonine, histidine, carnitine, taurine, glycine, alanine, serine cystine, thyroxine aspartic acid, asparagine glutamic acid glutamine hydroxylysine, proline, hydroxyproline and mixtures thereof.

37. A liquid nutritional formula according to claim 27, wherein said carbohydrate component is selected from the group consisting of hydrolyzed, intact, natural and chemically modified starches sourced from corn, tapioca, rice or potato in waxy or non-waxy forms; sugars such as glucose, fructose, lactose, sucrose, maltose, high fructose corn syrup, corn syrup solids; and mixtures thereof.
